(19) United States
(12) Patent Application Publication
(10) Pub. No.: US 2021/0246156 A1
(43) Pub. Date: Aug. 12, 2021

(54) HIGH-PURITY STEVIOL GLYCOSIDES

(71) Applicant: PURECIRCLE USA, INC., CHICAGO, IL (US)

(72) Inventors: Avetik MARKOSYAN, Yerevan (AM); Saravanan A/L RAMANDACH, Negeri Sembilan (MY); Mohamad Yin AFZAAL BIN HASIM, Kuala Lumpur (MY); Kristina CHKHAN, Kuala Lumpur (MY); Khairul NIZAM BIN NAWI, Negeri Sembilan (MY); Siew Yin CHOW, Selangor (MY)

(21) Appl. No.: 16/973,283

(22) PCT Filed: Jun. 7, 2019
(86) PCT No.: PCT/US2019/036125

§ 371 Date: Dec. 8, 2020

Related U.S. Application Data
(60) Provisional application No. 62/682,461, filed on Jun. 8, 2018.

(51) Int. Cl. C07H 15/256; C12P 19/56; A23L 2/60; A23L 27/30

(52) U.S. Cl. CPC C07H 15/256; C12P 19/56; C12R 2001/19; A23L 27/36; A23L 2/60

(57) ABSTRACT

Methods of preparing highly purified steviol glycosides, particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 are described. The methods include utilizing enzyme preparations and recombinant microorganisms for converting various staring compositions to target steviol glycosides. The highly purified steviol glycosides are useful as non-caloric sweetener, flavor enhancer, sweetness enhancer, and foaming suppressor in edible and chewable compositions such as any beverages, confectioneries, bakery products, cookies, and chewing gums.

### TECHNICAL FIELD

The present invention relates to a process for preparing compositions comprising steviol glycosides, including highly purified steviol glycoside compositions.

### SEQUENCE LISTING

The text file entitled “PC_78PROV.txt,” created on Jun. 8, 2018, having 19 kilobytes of data, and filed concurrently herewith, is hereby incorporated by reference in its entirety in this application.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 or a synthetic steviol glycoside.

In one embodiment, the target steviol glycoside is rebaudioside N2.

In one embodiment, the target steviol glycoside is rebaudioside O4.

In some preferred embodiments enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides. Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

The enzymes necessary for converting the starting composition to target steviol glycosides include the steviol biosynthesis enzymes, UDP-glucosyltransferases (UGTs), UDP-glycosyltransferases (hereinafter UGlyTs) and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

The UDP-glycosyltransferase can be any UDP-glycosyltransferase capable of adding at least one rhamnose unit to steviol and/or a steviol glycoside substrate to provide the target steviol glycoside.

As used hereinafter, the term “SuSy_AT”, unless specified otherwise, refers to sucrose synthase having amino-acid sequence “SEQ ID 1” as described in Example 1, or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 1 polypeptide as well as isolated nucleic acid molecules that code for those polypeptides.

As used hereinafter, the term “UGTSl2”, unless specified otherwise, refers to UDP-glucosyltransferase having amino-acid sequence “SEQ ID 2” as described in Example 1 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide as well as isolated nucleic acid molecules that code for those polypeptides.

As used hereinafter, the term “UGT76G1”, unless specified otherwise, refers to UDP-glucosyltransferase having amino-acid sequence “SEQ ID 3” as described in Example 1 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide as well as isolated nucleic acid molecules that code for those polypeptides.

As used hereinafter, the term “UGlyT91C1”, unless specified otherwise, refers to UDP-glycosyltransferase having amino-acid sequence “SEQ ID 4” as described in Example 1 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 4 polypeptide as well as isolated nucleic acid molecules that code for those polypeptides.

In one embodiment, steviol biosynthesis enzymes, UDP-glucosyltransferases and UDP-glycosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTSl2, EUGT11 and UGTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGTs.

In one embodiment, the UDP-glycosyltransferase is selected from group including UGlyT91C1 and UGlyTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGlyTs.

In one embodiment, steviol biosynthesis enzymes, UGTs, UGlyTs, UDP-glucose recycling system and UDP-rhamnose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glycosyltransferase is any UDP-glycosyltransferase capable of adding at least one rhamnose unit to the existing glucose at C13 of any starting steviol glycoside to give a target steviol glycoside with at least one additional rhamnose bearing at least one alpha 1→2 rhamnopyranoside glycosidic linkage(s) at the newly formed glycosidic bond(s). In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1→6 glucopyranoside glycosidic linkage(s) at the newly formed glycosidic bond(s). In a particular embodiment, the UDP-glucosyltransferase is UGTSl2, or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolmonoside to form steviolbioside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside to form steviolbioside E. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In one embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside C to form dulcoside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside C to form dulcoside D. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside C to form dulcoside A. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolbioside D to form dulcoside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolbioside D to form rebaudioside B2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside D to form stevioside G. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolbioside E to form dulcoside D. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolbioside E to form rebaudioside B2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside E to form stevioside H. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to rubusoside to form dulcoside A. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rubusoside to form stevioside G. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside H. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside A. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside C to form rebaudioside C3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside C to form rebaudioside C. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to dulcoside D to form rebaudioside C3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to dulcoside D to form rebaudioside C4. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to rebaudioside B2 to form rebaudioside C3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside B2 to form rebaudioside A4. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to dulcoside A to form rebaudioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to dulcoside A to form rebaudioside C4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside A to form rebaudioside C5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside A to form rebaudioside C6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside G to form rebaudioside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside G to form rebaudioside A4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside G to form rebaudioside E4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside G to form rebaudioside E6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside H to form rebaudioside C4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside H to form rebaudioside A4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside H to form rebaudioside E5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside H to form rebaudioside E7. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside A to form rebaudioside C5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside B to form rebaudioside C6. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside B to form rebaudioside E6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E7. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to rebaudioside C3 to form rebaudioside H2. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to rebaudioside C to form rebaudioside H2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C to form rebaudioside K In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C to form rebaudioside H4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C4 to form rebaudioside H2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C4 to form rebaudioside H3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C4 to form rebaudioside H5. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside A4 to form rebaudioside H2. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside A4 to form rebaudioside D5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside A4 to form rebaudioside D6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C5 to form rebaudioside K In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C5 to form rebaudioside H3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C5 to form rebaudioside H6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside E4 to form rebaudioside K In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E4 to form rebaudioside D5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D7. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside E5 to form rebaudioside H3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E5 to form rebaudioside D5. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E5 to form rebaudioside D8. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C6 to form rebaudioside H4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C6 to form rebaudioside H5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C6 to form rebaudioside H6. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E6 to form rebaudioside H4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E6 to form rebaudioside D6. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E6 to form rebaudioside D7. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E7 to form rebaudioside H5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E7 to form rebaudioside D6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E7 to form rebaudioside D8. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E3 to form rebaudioside H6. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E3 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E3 to form rebaudioside D8. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H2 to form rebaudioside N3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H2 to form rebaudioside N4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside K to form rebaudioside N3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside K to form rebaudioside N2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H3 to form rebaudioside N3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H3 to form rebaudioside N5. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside D5 to form rebaudioside N3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D5 to form rebaudioside M3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H4 to form rebaudioside N4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H4 to form rebaudioside N2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H5 to form rebaudioside N4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H5 to form rebaudioside N5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D6 to form rebaudioside N4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D6 to form rebaudioside M3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H6 to form rebaudioside N2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H6 to form rebaudioside N5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D7 to form rebaudioside N2. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D7 to form rebaudioside M3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D8 to form rebaudioside N5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D8 to form rebaudioside M3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside N3 to form rebaudioside O4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside N4 to form rebaudioside O4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside N2 to form rebaudioside O4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside N5 to form rebaudioside K4. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside M3 to form rebaudioside O4. In a particular embodiment, the UDP-glycosyltransferase is UGlyT91C1, or a UGlyT having >85% amino-acid sequence identity with UGlyT91C1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose.

Optionally, the method of the present invention further comprises recycling UDP to provide UDP-rhamnose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glycosyltransferase and UDP-rhamnose.

In one embodiment, the recycling catalyst is sucrose synthase SuSy_At or a sucrose synthase having >85% amino-acid sequence identity with SuSy_At.

In one embodiment, the recycling substrate for UDP-glucose recycling catalyst is sucrose.

In one embodiment, the recycling substrate for UDP-rhamnose recycling catalyst is a molecule comprising rhamnosyl residue.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside N2. In another embodiment, the recipient steviol glycoside is rebaudioside O4.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

The target steviol glycoside can be in any polymorphic or amorphous form, including hydrates, solvates, anhydrous or combinations thereof.

Purified target steviol glycosides can be used in consumable products as a sweetener, flavor modifier, flavor with modifying properties and/or foaming suppressor. Suitable consumer products include, but are not limited to, food, beverages, pharmaceutical compositions, tobacco products, nutraceutical compositions, oral hygiene compositions, and cosmetic compositions.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIGS. 1a and 1b show the chemical structure of rebaudioside N2. FIGS. 1c and 1d show the chemical structure of rebaudioside O4. FIG. 1e shows the chemical structure of a steviol glycoside with an aglycone structure.

FIG. 2a thru FIG. 21 show the pathways of producing rebaudioside N2, rebaudioside O4 and various steviol glycosides from steviol and the various intermediary steviol glycosides.

FIG. 3a shows the biocatalytic production of rebaudioside N2 from rebaudioside C using the enzymes UGTSl2 and UGT76G1 and concomitant recycling of UDP to UDP-glucose via sucrose synthase SuSy_At. FIG. 3b shows the biocatalytic production of rebaudioside O4 from rebaudioside C using the enzymes UGTSl2 and UGT76G1 and concomitant recycling of UDP to UDP-glucose via sucrose synthase SuSy_At.

FIG. 4 shows the HPLC chromatogram of rebaudioside C. The peak with retention time of 24.535 minutes corresponds to rebaudioside C.

FIG. 5 shows the HPLC chromatogram of the product of the biocatalytic production of rebaudioside N2 and rebaudioside O4 from rebaudioside C. The peaks with retention time of 11.317 minutes and 8.603 minutes correspond to rebaudioside N2 and rebaudioside O4, respectively. The peak at 12.567 minutes corresponds to rebaudioside K.

FIG. 6a shows the HPLC chromatogram of rebaudioside N2 after purification by HPLC. The peak with retention time of 10.745 minutes corresponds to rebaudioside N2. FIG. 6b shows the HPLC chromatogram of rebaudioside O4 after purification by HPLC. The peak with retention time of 8.185 minutes corresponds to rebaudioside O4.

FIGS. 7a and 7b show the 1H NMR spectrum of rebaudioside N2 and rebaudioside O4 (500 MHz, pyridine-d5), respectively.

FIGS. 8a and 8b show the HSQC spectrum of rebaudioside N2 and rebaudioside O4 (500 MHz, pyridine-d5), respectively.

FIGS. 9a and 9b show the H,H COSY spectrum of rebaudioside N2 and rebaudioside O4 (500 MHz, pyridine-d5), respectively.

FIGS. 10a and 10b show the HMBC spectrum of rebaudioside N2 and rebaudioside O4 (500 MHz, pyridine-d5), respectively.

FIGS. 11a and 11b show the HSQC-TOCSY spectrum of rebaudioside N2 and rebaudioside O4 (500 MHz, pyridine-d5), respectively.

FIGS. 12a and 12b show the LC chromatogram and mass spectrum of rebaudioside N2 respectively.

FIGS. 13a and 13b show the LC chromatogram and mass spectrum of rebaudioside O4 respectively.

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 or a synthetic steviol glycoside from various starting compositions.

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells comprising one or more enzyme, capable of single or multiple step chemical transformations on organic compounds. Biocatalysis processes include fermentation, biosynthesis, bioconversion and biotransformation processes. Both isolated enzyme, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 synthetic steviol glycosides, e.g. enzymatically glucosylated steviol glycosides and combinations thereof.

### Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 or other glycoside of steviol occurring in Stevia rebaudiana plant, synthetic steviol glycosides, e.g. enzymatically glucosylated steviol glycosides and combinations thereof.

In one embodiment, the starting composition is steviol.

In another embodiment, the starting composition steviol glycoside is steviolmonoside.

In yet another embodiment, the starting composition steviol glycoside is steviolmonoside A.

In another embodiment, the starting composition steviol glycoside is steviolbioside B.

In another embodiment, the starting composition steviol glycoside is steviolbioside C.

In another embodiment, the starting composition steviol glycoside is steviolbioside D.

In another embodiment, the starting composition steviol glycoside is steviolbioside E.

In another embodiment, the starting composition steviol glycoside is rubusoside.

In another embodiment, the starting composition steviol glycoside is dulcoside A.

In another embodiment, the starting composition steviol glycoside is dulcoside C.

In another embodiment, the starting composition steviol glycoside is dulcoside D.

In another embodiment, the starting composition steviol glycoside is stevioside A.

In another embodiment, the starting composition steviol glycoside is stevioside B.

In another embodiment, the starting composition steviol glycoside is stevioside C.

In another embodiment, the starting composition steviol glycoside is stevioside G.

In another embodiment, the starting composition steviol glycoside is stevioside H.

In another embodiment, the starting composition steviol glycoside is rebaudioside B2.

In another embodiment, the starting composition steviol glycoside is rebaudioside B2.

In another embodiment, the starting composition steviol glycoside is rebaudioside A4.

In another embodiment, the starting composition steviol glycoside is rebaudioside C.

In another embodiment, the starting composition steviol glycoside is rebaudioside C3.

In another embodiment, the starting composition steviol glycoside is rebaudioside C4.

In another embodiment, the starting composition steviol glycoside is rebaudioside C5.

In another embodiment, the starting composition steviol glycoside is rebaudioside C6.

In another embodiment, the starting composition steviol glycoside is rebaudioside E3.

In another embodiment, the starting composition steviol glycoside is rebaudioside E4.

In another embodiment, the starting composition steviol glycoside is rebaudioside E5.

In another embodiment, the starting composition steviol glycoside is rebaudioside E6.

In another embodiment, the starting composition steviol glycoside is rebaudioside E7.

In another embodiment, the starting composition steviol glycoside is rebaudioside D5.

In another embodiment, the starting composition steviol glycoside is rebaudioside D6.

In another embodiment, the starting composition steviol glycoside is rebaudioside D7.

In another embodiment, the starting composition steviol glycoside is rebaudioside D8.

In another embodiment, the starting composition steviol glycoside is rebaudioside H2.

In another embodiment, the starting composition steviol glycoside is rebaudioside H3.

In another embodiment, the starting composition steviol glycoside is rebaudioside H4.

In another embodiment, the starting composition steviol glycoside is rebaudioside H5.

In another embodiment, the starting composition steviol glycoside is rebaudioside H6.

In another embodiment, the starting composition steviol glycoside is rebaudioside K.

In another embodiment, the starting composition steviol glycoside is rebaudioside N2.

In another embodiment, the starting composition steviol glycoside is rebaudioside N3.

In another embodiment, the starting composition steviol glycoside is rebaudioside N4.

In another embodiment, the starting composition steviol glycoside is rebaudioside N5.

In another embodiment, the starting composition steviol glycoside is rebaudioside M3.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In another embodiment, the starting composition is rhamnose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4, or other glycoside of steviol occurring in Stevia rebaudiana plant, synthetic steviol glycosides, e.g. enzymatically glucosylated steviol glycosides and combinations thereof

In one embodiment, the target steviol glycoside is steviolmonoside.

In another embodiment, the target steviol glycoside is steviolmonoside A.

In another embodiment, the target steviol glycoside is steviolbioside A.

In another embodiment, the target steviol glycoside is steviolbioside B.

In another embodiment, the target steviol glycoside is steviolbioside C,

In another embodiment, the target steviol glycoside is steviolbioside D.

In another embodiment, the target steviol glycoside is steviolbioside E.

In another embodiment, the target steviol glycoside is rubusoside.

In another embodiment, the target steviol glycoside is dulcoside A.

In another embodiment, the target steviol glycoside is dulcoside C.

In another embodiment, the target steviol glycoside is dulcoside D.

In another embodiment, the target steviol glycoside is stevioside A.

In another embodiment, the target steviol glycoside is stevioside B.

In another embodiment, the target steviol glycoside is stevioside C.

In another embodiment, the target steviol glycoside is stevioside G.

In another embodiment, the target steviol glycoside is stevioside H.

In another embodiment, the target steviol glycoside is rebaudioside B2.

In another embodiment, the target steviol glycoside is rebaudioside A4.

In another embodiment, the target steviol glycoside is rebaudioside C.

In another embodiment, the target steviol glycoside is rebaudioside C3.

In another embodiment, the target steviol glycoside is rebaudioside C4.

In another embodiment, the target steviol glycoside is rebaudioside C5.

In another embodiment, the target steviol glycoside is rebaudioside C6.

In another embodiment, the target steviol glycoside is rebaudioside E3.

In another embodiment, the target steviol glycoside is rebaudioside E4.

In another embodiment, the target steviol glycoside is rebaudioside E5.

In another embodiment, the target steviol glycoside is rebaudioside E6.

In another embodiment, the target steviol glycoside is rebaudioside E7.

In another embodiment, the target steviol glycoside is rebaudioside D5.

In another embodiment, the target steviol glycoside is rebaudioside D6.

In another embodiment, the target steviol glycoside is rebaudioside D7.

In another embodiment, the target steviol glycoside is rebaudioside D8.

In another embodiment, the target steviol glycoside is rebaudioside H2.

In another embodiment, the target steviol glycoside is rebaudioside H3.

In another embodiment, the target steviol glycoside is rebaudioside H4.

In another embodiment, the target steviol glycoside is rebaudioside H5.

In another embodiment, the target steviol glycoside is rebaudioside H6.

In another embodiment, the target steviol glycoside is rebaudioside K.

In another embodiment, the target steviol glycoside is rebaudioside N2.

In another embodiment, the target steviol glycoside is rebaudioside N3

In another embodiment, the target steviol glycoside is rebaudioside N4.

In another embodiment, the target steviol glycoside is rebaudioside N5.

In another embodiment, the target steviol glycoside is rebaudioside M3.

In another embodiment, the target steviol glycoside is rebaudioside O4.

The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside A.

In one embodiment, the present invention is a biocatalytic process for the production of steviolbioside B.

In one embodiment, the present invention is a biocatalytic process for the production of steviolbioside C.

In one embodiment, the present invention is a biocatalytic process for the production of steviolbioside D.

In one embodiment, the present invention is a biocatalytic process for the production of steviolbioside E.

In one embodiment, the present invention is a biocatalytic process for the production of rubusoside.

In one embodiment, the present invention is a biocatalytic process for the production of dulcoside A.

In one embodiment, the present invention is a biocatalytic process for the production of C.

In one embodiment, the present invention is a biocatalytic process for the production of dulcoside D.

In one embodiment, the present invention is a biocatalytic process for the production of stevioside A.

In one embodiment, the present invention is a biocatalytic process for the production of stevioside B.

In one embodiment, the present invention is a biocatalytic process for the production of stevioside C.

In one embodiment, the present invention is a biocatalytic process for the production of stevioside G.

In one embodiment, the present invention is a biocatalytic process for the production of stevioside H.

In one embodiment, the present invention is a biocatalytic process for the production of rebaudioside B2.

In one embodiment, the present invention is a biocatalytic process for the production of rebaudioside A4.

In one embodiment, the present invention is a biocatalytic process for the production of rebaudioside C.

In one embodiment, the present invention is a biocatalytic process for the production of rebaudioside C3.

In one embodiment, the present invention is a biocatalytic process for the production of rebaudioside C4.

In one embodiment, the present invention is a biocatalytic process for the production of rebaudioside C5.

In one embodiment, the present invention is a biocatalytic process for the production of rebaudioside C6.

In one embodiment, the present invention is a biocatalytic process for the production of rebaudioside E3.

In one embodiment, the present invention is a biocatalytic process for the production of rebaudioside E4.

In one embodiment, the present invention is a biocatalytic process for the production of rebaudioside E5.

In one embodiment, the present invention is a biocatalytic process for the production of rebaudioside E6.

In one embodiment, the present invention is a biocatalytic process for the production of rebaudioside E7.

In one embodiment, the present invention is a biocatalytic process for the production of rebaudioside D5.

In one embodiment, the present invention is a biocatalytic process for the production of rebaudioside D6.

In one embodiment, the present invention is a biocatalytic process for the production of rebaudioside D7.

In one embodiment, the present invention is a biocatalytic process for the production of rebaudioside D8.

In one embodiment, the present invention is a biocatalytic process for the production of rebaudioside H2.

In one embodiment, the present invention is a biocatalytic process for the production of rebaudioside H3.

In one embodiment, the present invention is a biocatalytic process for the production of rebaudioside H4.

In one embodiment, the present invention is a biocatalytic process for the production of rebaudioside H5.

In one embodiment, the present invention is a biocatalytic process for the production of rebaudioside H6.

In one embodiment, the present invention is a biocatalytic process for the production of rebaudioside K.

In one embodiment, the present invention is a biocatalytic process for the production of rebaudioside N2.

In one embodiment, the present invention is a biocatalytic process for the production of rebaudioside N3

In one embodiment, the present invention is a biocatalytic process for the production of rebaudioside N4.

In one embodiment, the present invention is a biocatalytic process for the production of rebaudioside N5.

In one embodiment, the present invention is a biocatalytic process for the production of rebaudioside M3.

In one embodiment, the present invention is a biocatalytic process for the production of rebaudioside O4.

In a particular embodiment, the present invention provides for the biocatalytic process for the production of rebaudioside N2 from a starting composition comprising rebaudioside C and UDP-glucose.

In another particular embodiment, the present invention provides for the biocatalytic process for the production of rebaudioside O4 from a starting composition comprising rebaudioside C and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is reb N2, the process described herein provides a composition having greater than about 90% reb N2 content by weight on a dried basis. In another particular embodiment, when the target steviol glycoside is reb N2, the process described herein provides a composition comprising greater than about 95% reb N2 content by weight on a dried basis.

In another embodiment, when the target steviol glycoside is reb O4, the process described herein provides a composition having greater than about 90% reb O4 content by weight on a dried basis. In another particular embodiment, when the target steviol glycoside is reb O4, the process described herein provides a composition comprising greater than about 95% reb O4 content by weight on a dried basis.

### Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

Suitable enzymes for converting the starting composition to target steviol glycosides include, but are not limited to, the steviol biosynthesis enzymes, UDP-glucosyltransferases (UGTs) and UDP glycosyltransferases (UGlyTs). Optionally it may include UDP recycling enzyme(s).

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

The UDP-glycosyltransferase can be any UDP-glycosyltransferase capable of adding at least one rhamnose unit to steviol and/or a steviol glycoside substrate to provide the target steviol glycoside.

In one embodiment, steviol biosynthesis enzymes, UDP-glucosyltransferases and UDP-glycosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTSl2, EUGT11 and UGTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGTs.

In one embodiment, the UDP-glycosyltransferase is selected from group including UGlyT91C1 and UGlyTs having substantial (>85%, >86%, >87%, >88%, >89%, >90%, >91%, >92%, >93%, >94%, >95%, >96%, >97%, >98%, >99%) amino-acid sequence identity to these polypeptides as well as isolated nucleic acid molecules that code for these UGlyTs.

In one embodiment, steviol biosynthesis enzymes, UGTs, UGlyTs, UDP-glucose recycling system and UDP-rhamnose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTSl2, or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glycosyltransferase is any UDP-glycosyltransferase capable of adding at least one rhamnose unit to the existing glucose at C13 of any starting steviol glycoside to give a target steviol glycoside with at least one additional rhamnose bearing at least one alpha 1->2 rhamnopyranoside glycosidic linkage(s) at the newly formed glycosidic bond(s). In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1->6 glucopyranoside glycosidic linkage(s) at the newly formed glycosidic bond(s). In a particular embodiment, the UDP-glucosyltransferase is UGTSl2, or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolmonoside to form steviolbioside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolmonoside to form steviolbioside D. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside to form steviolbioside E. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In one embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside C to form dulcoside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside C to form dulcoside D. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside C to form dulcoside A. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolbioside D to form dulcoside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolbioside D to form rebaudioside B2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside D to form stevioside G. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to steviolbioside E to form dulcoside D. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to steviolbioside E to form rebaudioside B2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolbioside E to form stevioside H. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to rubusoside to form dulcoside A. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rubusoside to form stevioside G. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside H. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside A. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In one embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside C to form rebaudioside C3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside C to form rebaudioside C. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to dulcoside D to form rebaudioside C3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to dulcoside D to form rebaudioside C4. In a particular embodiment, the UDP-glucosyltransferase is UGT74G1 or a UGT having >85% amino-acid sequence identity with UGT74G1.

In another embodiment, the UDP-glycosyltransferase is any UDP-glycosyltransferase capable of adding at least one rhamnose unit to rebaudioside B2 to form rebaudioside C3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside B2 to form rebaudioside A4. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to dulcoside A to form rebaudioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to dulcoside A to form rebaudioside C4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside A to form rebaudioside C5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to dulcoside A to form rebaudioside C6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside G to form rebaudioside C. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside G to form rebaudioside A4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside G to form rebaudioside E4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside G to form rebaudioside E6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside H to form rebaudioside C4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside H to form rebaudioside A4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside H to form rebaudioside E5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside H to form rebaudioside E7. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside A to form rebaudioside C5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside A to form rebaudioside E4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A to form rebaudioside E5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to stevioside B to form rebaudioside C6. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to stevioside B to form rebaudioside E6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside B to form rebaudioside E7. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to rebaudioside C3 to form rebaudioside H2. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to rebaudioside C to form rebaudioside H2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C to form rebaudioside K In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C to form rebaudioside H4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C4 to form rebaudioside H2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C4 to form rebaudioside H3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C4 to form rebaudioside H5. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside A4 to form rebaudioside H2. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside A4 to form rebaudioside D5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside A4 to form rebaudioside D6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C5 to form rebaudioside K In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C5 to form rebaudioside H3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C5 to form rebaudioside H6. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside E4 to form rebaudioside K In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E4 to form rebaudioside D5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E4 to form rebaudioside D7. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside E5 to form rebaudioside H3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E5 to form rebaudioside D5. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E5 to form rebaudioside D8. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C6 to form rebaudioside H4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside C6 to form rebaudioside H5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside C6 to form rebaudioside H6. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E6 to form rebaudioside H4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E6 to form rebaudioside D6. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E6 to form rebaudioside D7. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E7 to form rebaudioside H5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E7 to form rebaudioside D6. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E7 to form rebaudioside D8. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside E3 to form rebaudioside H6. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside E3 to form rebaudioside D7. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside E3 to form rebaudioside D8. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H2 to form rebaudioside N3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H2 to form rebaudioside N4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside K to form rebaudioside N3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside K to form rebaudioside N2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H3 to form rebaudioside N3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H3 to form rebaudioside N5. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside D5 to form rebaudioside N3. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D5 to form rebaudioside M3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H4 to form rebaudioside N4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H4 to form rebaudioside N2. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H5 to form rebaudioside N4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H5 to form rebaudioside N5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D6 to form rebaudioside N4. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D6 to form rebaudioside M3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside H6 to form rebaudioside N2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside H6 to form rebaudioside N5. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D7 to form rebaudioside N2. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D7 to form rebaudioside M3. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glycosyltransferase is any UDP-glycosyltransferase capable of adding at least one rhamnose unit to rebaudioside D8 to form rebaudioside N5. In a particular embodiment, the UDP-glycosyltransferase is UGlyT91C1, or a UGlyT having >85% amino-acid sequence identity with UGlyT91C1.

In another embodiment, the UDP-glucosyltransferase is any UDP-glucosyltransferase capable of adding at least one glucose unit to rebaudioside D8 to form rebaudioside M3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside N3 to form rebaudioside O4. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rebaudioside N4 to form rebaudioside O4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside N2 to form rebaudioside O4. In a particular embodiment, the UDP-glucosyltransferase is UGTSl2 or a UGT having >85% amino-acid sequence identity with UGTSl2. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside N5 to form rebaudioside K4. In a particular embodiment, the UDP-glucosyltransferase is UGT76G1 or a UGT having >85% amino-acid sequence identity with UGT76G1.

In another embodiment, the UDP-glycosyltransferase is any UDP-glycosyltransferase capable of adding at least one rhamnose unit to rebaudioside M3 to form rebaudioside O4. In a particular embodiment, the UDP-glycosyltransferase is UGlyT91C1, or a UGlyT having >85% amino-acid sequence identity with UGlyT91C1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

Optionally, the method of the present invention further comprises recycling UDP to provide UDP-rhamnose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glycosyltransferase and UDP-rhamnose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside N2. In another embodiment, the recipient steviol glycoside is rebaudioside O4.

In another embodiment, the UDP-glucosyltransferase capable of adding at least one glucose unit to starting composition steviol glycoside has >85% amino-acid sequence identity with UGTs selected from the following listing of GenInfo identifier numbers, preferably from the group presented in Table 1, and Table 2.

| | | | | | |
|---|---|---|---|---|---|
| 397567 | 30680413 | 115480946 | 147798902 | 218193594 | 225443294 |
| 454245 | 32816174 | 116310259 | 147811764 | 218193942 | 225444853 |
| 1359905 | 32816178 | 116310985 | 147827151 | 219885307 | 225449296 |
| 1685003 | 34393978 | 116788066 | 147836230 | 222615927 | 225449700 |
| 1685005 | 37993665 | 116788606 | 147839909 | 222619587 | 225454338 |
| 2191136 | 37993671 | 116789315 | 147846163 | 222623142 | 225454340 |
| 2501497 | 37993675 | 119394507 | 147855977 | 222625633 | 225454342 |
| 2911049 | 39104603 | 119640480 | 148905778 | 222625635 | 225454473 |
| 4218003 | 41469414 | 122209731 | 148905999 | 222636620 | 225454475 |
| 4314356 | 41469452 | 125526997 | 148906835 | 222636621 | 225458362 |
| 13492674 | 42566366 | 125534279 | 148907340 | 222636628 | 225461551 |
| 13492676 | 42570280 | 125534461 | 148908935 | 222636629 | 225461556 |
| 15217773 | 42572855 | 125540090 | 148909182 | 224053242 | 225461558 |
| 15217796 | 44890129 | 125541516 | 148909920 | 224053386 | 225469538 |
| 15223396 | 46806235 | 125545408 | 148910082 | 224055535 | 225469540 |
| 15223589 | 50284482 | 125547340 | 148910154 | 224056138 | 226316457 |
| 15227766 | 51090402 | 125547520 | 148910612 | 224056160 | 226492603 |
| 15230017 | 51090594 | 125554547 | 148910769 | 224067918 | 226494221 |
| 15231757 | 52839682 | 125557592 | 156138791 | 224072747 | 226495389 |
| 15234056 | 56550539 | 125557593 | 156138797 | 224080189 | 226495945 |
| 15234195 | 62734263 | 125557608 | 156138799 | 224091845 | 226502400 |
| 15234196 | 62857204 | 125559566 | 156138803 | 224094703 | 226507980 |
| 15238503 | 62857206 | 125563266 | 165972256 | 224100653 | 226531147 |
| 15239523 | 62857210 | 125571055 | 168016721 | 224100657 | 226532094 |
| 15239525 | 62857212 | 125579728 | 171674071 | 224101569 | 238477377 |
| 15239543 | 75265643 | 125588307 | 171906258 | 224103105 | 240254512 |
| 15239937 | 75285934 | 125589492 | 183013901 | 224103633 | 242032615 |
| 15240305 | 75288884 | 125599469 | 183013903 | 224103637 | 242032621 |
| 15240534 | 77550661 | 125601477 | 186478321 | 224109218 | 242038423 |
| 15982889 | 77556148 | 126635837 | 187373030 | 224114583 | 242043290 |
| 18086351 | 82791223 | 126635845 | 187373042 | 224116284 | 242044836 |
| 18418378 | 83778990 | 126635847 | 190692175 | 224120552 | 242051252 |
| 18418380 | 89953335 | 126635863 | 194701936 | 224121288 | 242056217 |
| 18418382 | 110741436 | 126635867 | 195620060 | 224121296 | 242056219 |
| 19743740 | 110743955 | 126635883 | 209954691 | 224121300 | 242056663 |
| 19911201 | 115438196 | 126635887 | 209954719 | 224130358 | 242059339 |
| 20149064 | 115438785 | 133874210 | 209954725 | 224140703 | 242059341 |
| 20260654 | 115441237 | 133874212 | 209954733 | 224143404 | 242060922 |
| 21435782 | 115454819 | 145358033 | 210063105 | 224143406 | 242067411 |
| 21553613 | 115456047 | 147772508 | 210063107 | 224144306 | 242067413 |
| 21593514 | 115457492 | 147776893 | 212275846 | 224285244 | 242076258 |
| 22759895 | 115459312 | 147776894 | 216296854 | 225431707 | 242076396 |
| 23955910 | 115464719 | 147776895 | 217074506 | 225435532 | 242084750 |
| 26452040 | 115471069 | 147786916 | 218185693 | 225436321 | 242091005 |
| 28393204 | 115471071 | 147798900 | 218187075 | 225440041 | 242095206 |
| 30679796 | 115474009 | 147798901 | 218189427 | 225441116 | 242345159 |
| 242345161 | 297724601 | 326492035 | 356523945 | 357140904 | 359486938 |
| 255536859 | 297725463 | 326493430 | 356523957 | 357165849 | 359487055 |
| 255538228 | 297728331 | 326500410 | 356523959 | 357165852 | 359488135 |
| 255541676 | 297738632 | 326506816 | 356523961 | 357168415 | 359488708 |
| 255547075 | 297745347 | 326507826 | 356523963 | 357437837 | 359493630 |
| 255552620 | 297745348 | 326508394 | 356524387 | 357442755 | 359493632 |
| 255552622 | 297795735 | 326509445 | 356524403 | 357442757 | 359493634 |
| 255555343 | 297796253 | 326511261 | 356527181 | 357445729 | 359493636 |
| 255555361 | 297796257 | 326511866 | 356533209 | 357445731 | 359493815 |
| 255555363 | 297796261 | 326512412 | 356533852 | 357445733 | 359495856 |
| 255555365 | 297797587 | 326517673 | 356534718 | 357446799 | 359495858 |
| 255555369 | 297798502 | 326518800 | 356535480 | 357446805 | 359495869 |
| 255555373 | 297799226 | 326521124 | 356542996 | 357452779 | 359495871 |
| 255555377 | 297805988 | 326525567 | 356543136 | 357452781 | 359497638 |
| 255556812 | 297807499 | 326525957 | 356543932 | 357452783 | 359807261 |
| 255556818 | 297809125 | 326526607 | 356549841 | 357452787 | 374256637 |
| 255563008 | 297809127 | 326527141 | 356549843 | 357452789 | 377655465 |
| 255564074 | 297811403 | 326530093 | 356554358 | 357452791 | 378405177 |
| 255564531 | 297820040 | 326534036 | 356554360 | 357452797 | 378829085 |
| 255572878 | 297821483 | 326534312 | 356558606 | 357452799 | 387135070 |
| 255577901 | 297825217 | 332071132 | 356560333 | 357470367 | 387135072 |
| 255583249 | 297832276 | 339715876 | 356560599 | 357472193 | 387135078 |
| 255583253 | 297832280 | 342306012 | 356560749 | 357472195 | 387135092 |
| 255583255 | 297832518 | 342306016 | 356566018 | 357474295 | 387135094 |
| 255585664 | 297832520 | 343457675 | 356566169 | 357474493 | 387135098 |
| 255585666 | 297840825 | 343457677 | 356566173 | 357474497 | 387135100 |
| 255634688 | 297840827 | 350534960 | 356567761 | 357474499 | 387135134 |
| 255644801 | 297847402 | 356498085 | 356574704 | 357490035 | 387135136 |
| 255645821 | 297849372 | 356499771 | 356576401 | 357493567 | 387135174 |
| 255647456 | 300078590 | 356499777 | 356577660 | 357497139 | 387135176 |
| 255648275 | 300669727 | 356499779 | 357114993 | 357497581 | 387135184 |
| 260279126 | 302142947 | 356501328 | 357115447 | 357497671 | 387135186 |
| 260279128 | 302142948 | 356502523 | 357115451 | 357500579 | 387135188 |
| 261343326 | 302142950 | 356503180 | 357115453 | 357504663 | 387135190 |
| 283132367 | 302142951 | 356503184 | 357116080 | 357504691 | 387135192 |
| 283362112 | 302765302 | 356503295 | 357116928 | 357504699 | 387135194 |
| 289188052 | 302796334 | 356504436 | 357117461 | 357504707 | 387135282 |
| 295841350 | 302811470 | 356504523 | 357117463 | 357505859 | 387135284 |
| 296088529 | 302821107 | 356504765 | 357117829 | 357510851 | 387135294 |
| 296090415 | 302821679 | 356511113 | 357117839 | 357516975 | 387135298 |
| 296090524 | 319759260 | 356515120 | 357125059 | 359477003 | 387135300 |
| 296090526 | 319759266 | 356517088 | 357126015 | 359477998 | 387135302 |
| 297599503 | 320148814 | 356520732 | 357134488 | 359478043 | 387135304 |
| 297601531 | 326489963 | 356522586 | 357135657 | 359478286 | 387135312 |
| 297611791 | 326490273 | 356522588 | 357138503 | 359484299 | 387135314 |
| 297722841 | 326491131 | 356522590 | 357139683 | 359486936 | 387135316 |
| 387135318 | 449440433 | 460376293 | 460413408 | 462423864 | 475546199 |
| 387135320 | 449445896 | 460378310 | 460416351 | 470101924 | 475556485 |
| 387135322 | 449446454 | 460380744 | 462394387 | 470102280 | 475559699 |
| 387135324 | 449447657 | 460381726 | 462394433 | 470102858 | 475578293 |
| 387135326 | 449449002 | 460382093 | 462394557 | 470104211 | 475591753 |
| 387135328 | 449449004 | 460382095 | 462395646 | 470104264 | 475593742 |
| 388493506 | 449449006 | 460382754 | 462395678 | 470104266 | 475612072 |
| 388495496 | 449451379 | 460384935 | 462396388 | 470106317 | 475622476 |
| 388498446 | 449451589 | 460384937 | 462396389 | 470106357 | 475622507 |
| 388499220 | 449451591 | 460385076 | 462396419 | 470115448 | 475623787 |
| 388502176 | 449451593 | 460385872 | 462396542 | 470130404 | 482550481 |
| 388517521 | 449453712 | 460386018 | 462397507 | 470131550 | 482550499 |
| 388519407 | 449453714 | 460389217 | 462399998 | 470136482 | 482550740 |
| 388521413 | 449453716 | 460394872 | 462400798 | 470136484 | 482550999 |
| 388827901 | 449453732 | 460396139 | 462401217 | 470136488 | 482552352 |
| 388827903 | 449457075 | 460397862 | 462402118 | 470136492 | 482554970 |
| 388827907 | 449467555 | 460397864 | 462402237 | 470137933 | 482555336 |
| 388827909 | 449468742 | 460398541 | 462402284 | 470137937 | 482555478 |
| 388827913 | 449495638 | 460403139 | 462402416 | 470140422 | 482556454 |
| 393887637 | 449495736 | 460403141 | 462404228 | 470140426 | 482557289 |
| 393887646 | 449499880 | 460403143 | 462406358 | 470140908 | 482558462 |
| 393887649 | 449502786 | 460403145 | 462408262 | 470141232 | 482558508 |
| 393990627 | 449503471 | 460405998 | 462409325 | 470142008 | 482558547 |
| 397746860 | 449503473 | 460407578 | 462409359 | 470142010 | 482561055 |
| 397789318 | 449515857 | 460407590 | 462409777 | 470142012 | 482561555 |
| 413924864 | 449518643 | 460409128 | 462411467 | 470143607 | 482562795 |
| 414590349 | 449519559 | 460409134 | 462414311 | 470143939 | 482562850 |
| 414590661 | 449522783 | 460409136 | 462414416 | 470145404 | 482565074 |
| 414591157 | 449524530 | 460409459 | 462414476 | 473923244 | 482566269 |
| 414879558 | 449524591 | 460409461 | 462415526 | 474114354 | 482566296 |
| 414879559 | 449528823 | 460409463 | 462415603 | 474143634 | 482566307 |
| 414879560 | 449528825 | 460409465 | 462415731 | 474202268 | 482568689 |
| 414888074 | 449534021 | 460409467 | 462416307 | 474299266 | 482570049 |
| 431812559 | 460365546 | 460410124 | 462416920 | 474363119 | 482570572 |
| 449432064 | 460366882 | 460410126 | 462416922 | 474366157 | 482575121 |
| 449432066 | 460369823 | 460410128 | 462416923 | 474429346 | |
| 449433069 | 460369829 | 460410130 | 462416924 | 475432777 | |
| 449436944 | 460369831 | 460410132 | 462417401 | 475473002 | |
| 449438665 | 460369833 | 460410134 | 462419769 | 475489790 | |
| 449438667 | 460370755 | 460410213 | 462420317 | 475511330 | |
| 449440431 | 460374714 | 460411200 | 462423366 | 475516200 | |

TABLE 1

| GI number | Accession | Origin |
|---|---|---|
| 190692175 | ACE87855.1 | Stevia rebaudiana |
| 41469452 | AAS07253.1 | Oryza sativa |
| 62857204 | BAD95881.1 | Ipomoea nil |
| 62857206 | BAD95882.1 | Ipomoea purperea |
| 56550539 | BAD77944.1 | Bellis perennis |
| 115454819 | NP_001051010.1 | Oryza sativa Japonica Group |
| 115459312 | NP_001053256.1 | Oryza sativa Japonica Group |
| 115471069 | NP_001059133.1 | Oryza sativa Japonica Group |
| 115471071 | NP_001059134.1 | Oryza sativa Japonica Group |
| 116310985 | CAH67920.1 | Oryza sativa Indica Group |
| 116788066 | ABK24743.1 | Picea sitchensis |
| 122209731 | Q2V6J9.1 | Fragaria × ananassa |
| 125534461 | EAY81009.1 | Oryza sativa Indica Group |
| 125559566 | EAZ05102.1 | Oryza sativa Indica Group |
| 125588307 | EAZ28971.1 | Oryza sativa Japonica Group |
| 148907340 | ABR16806.1 | Picea sitchensis |
| 148910082 | ABR18123.1 | Picea sitchensis |
| 148910612 | ABR18376.1 | Picea sitchensis |
| 15234195 | NP_194486.1 | Arabidopsis thaliana |
| 15239523 | NP_200210.1 | Arabidopsis thaliana |
| 15239937 | NP_196793.1 | Arabidopsis thaliana |
| 1685005 | AAB36653.1 | Nicotiana tabacum |
| 183013903 | ACC38471.1 | Medicago truncatula |
| 186478321 | NP_172511.3 | Arabidopsis thaliana |
| 187373030 | ACD03249.1 | Avena strigosa |
| 194701936 | ACF85052.1 | Zea mays |
| 19743740 | AAL92461.1 | Solanum lycopersicum |
| 212275846 | NP_001131009.1 | Zea mays |
| 222619587 | EEE55719.1 | Oryza sativa Japonica Group |
| 224055535 | XP_002298527.1 | Populus trichocarpa |
| 224101569 | XP_002334266.1 | Populus trichocarpa |
| 224120552 | XP_002318358.1 | Populus trichocarpa |
| 224121288 | XP_002330790.1 | Populus trichocarpa |
| 225444853 | XP_002281094 | Vitis vinifera |
| 225454342 | XP_002275850.1 | Vitis vinifera |
| 225454475 | XP_002280923.1 | Vitis vinifera |
| 225461556 | XP_002285222 | Vitis vinifera |
| 225469540 | XP_002270294.1 | Vitis vinifera |
| 226495389 | NP_001148083.1 | Zea mays |
| 226502400 | NP_001147674.1 | Zea mays |
| 238477377 | ACR43489.1 | Triticum aestivum |
| 240254512 | NP_565540.4 | Arabidopsis thaliana |
| 2501497 | Q43716.1 | Petunia × hybrida |
| 255555369 | XP_002518721.1 | Ricinus communis |
| 26452040 | BAC43110.1 | Arabidopsis thaliana |
| 296088529 | CBI37520.3 | Vitis vinifera |
| 297611791 | NP_001067852.2 | Oryza sativa Japonica Group |
| 297795735 | XP_002865752.1 | Arabidopsis lyrata subsp. lyrata |
| 297798502 | XP_002867135.1 | Arabidopsis lyrata subsp. lyrata |
| 297820040 | XP_002877903.1 | Arabidopsis lyrata subsp. lyrata |
| 297832276 | XP_002884020.1 | Arabidopsis lyrata subsp. lyrata |
| 302821107 | XP_002992218.1 | Selaginella moellendorffii |
| 30680413 | NP_179446.2 | Arabidopsis thaliana |
| 319759266 | ADV71369.1 | Pueraria montana var. lobata |
| 326507826 | BAJ86656.1 | Hordeum vulgare subsp. Vulgare |
| 343457675 | AEM37036.1 | Brassica rapa subsp. oleifera |
| 350534960 | NP_001234680.1 | Solanum lycopersicum |
| 356501328 | XP_003519477.1 | Glycine max |
| 356522586 | XP_003529927.1 | Glycine max |
| 356535480 | XP_003536273.1 | Glycine max |
| 357445733 | XP_003593144.1 | Medicago truncatula |
| 357452783 | XP_003596668.1 | Medicago truncatula |
| 357474493 | XP_003607531.1 | Medicago truncatula |
| 357500579 | XP_003620578.1 | Medicago truncatula |
| 357504691 | XP_003622634.1 | Medicago truncatula |
| 359477998 | XP_003632051.1 | Vitis vinifera |
| 359487055 | XP_002271587 | Vitis vinifera |
| 359495869 | XP_003635104.1 | Vitis vinifera |
| 387135134 | AFJ52948.1 | Linum usitatissimum |
| 387135176 | AFJ52969.1 | Linum usitatissimum |
| 387135192 | AFJ52977.1 | Linum usitatissimum |
| 387135282 | AFJ53022.1 | Linum usitatissimum |
| 387135302 | AFJ53032.1 | Linum usitatissimum |
| 387135312 | AFJ53037.1 | Linum usitatissimum |
| 388519407 | AFK47765.1 | Medicago truncatula |
| 393887646 | AFN26668.1 | Barbarea vulgaris subsp. arcuata |
| 414888074 | DAA64088.1 | Zea mays |
| 42572855 | NP_974524.1 | Arabidopsis thaliana |
| 449440433 | XP_004137989.1 | Cucumis sativus |
| 449446454 | XP_004140986.1 | Cucumis sativus |
| 449449004 | XP_004142255.1 | Cucumis sativus |
| 449451593 | XP_004143546.1 | Cucumis sativus |
| 449515857 | XP_004164964.1 | Cucumis sativus |
| 460382095 | XP_004236775.1 | Solanum lycopersicum |
| 460409128 | XP_004249992.1 | Solanum lycopersicum |
| 460409461 | XP_004250157.1 | Solanum lycopersicum |
| 460409465 | XP_004250159.1 | Solanum lycopersicum |
| 462396388 | EMJ02187.1 | Prunus persica |
| 462402118 | EMJ07675.1 | Prunus persica |
| 462409359 | EMJ14693.1 | Prunus persica |
| 462416923 | EMJ21660.1 | Prunus persica |
| 46806235 | BAD17459.1 | Oryza sativa Japonica Group |
| 470104266 | XP_004288529.1 | Fragaria vesca subsp. vesca |
| 470142008 | XP_004306714.1 | Fragaria vesca subsp. vesca |
| 475432777 | EMT01232.1 | Aegilops tauschii |
| 51090402 | BAD35324.1 | Oryza sativa Japonica Group |

TABLE 2

| | | | Internal |
|---|---|---|---|
| GI number | Accession | Origin | reference |
| 460409128 | XP.004249992.1 | Solanum lycopersicum | UGTSl |
| 460386018 | XP.004238697.1 | Solanum lycopersicum | — |
| 460409134 | XP.004249995.1 | Solanum lycopersicum | — |
| 460410132 | XP.004250485.1 | Solanum lycopersicum | UGTSl2 |
| 460410130 | XP.004250484.1 | Solanum lycopersicum | — |
| 460410128 | XP.004250483.1 | Solanum lycopersicum | — |
| 460378310 | XP.004234916.1 | Solanum lycopersicum | — |
| 209954733 | BAG80557.1 | Lycium barbarum | UGTLB |
| 209954725 | BAG80553.1 | Lycium barbarum | — |

One embodiment of the present invention is a microbial cell comprising an enzyme, i.e. an enzyme capable of converting the starting composition to the target steviol glycoside. Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microoganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

### Uses

Highly purified target glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 obtained according to this invention can be used “as-is” or in combination with other sweeteners, flavors, food ingredients and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla and combinations thereof.

Non-limiting examples of other food ingredients include, but are not limited to, acidulants, organic and amino acids, coloring agents, bulking agents, modified starches, gums, texturizers, preservatives, caffeine, antioxidants, emulsifiers, stabilizers, thickeners, gelling agents and combinations thereof.

Highly purified target glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 obtained according to this invention can be prepared in various polymorphic forms, including but not limited to hydrates, solvates, anhydrous, amorphous forms and combinations thereof.

Highly purified target glycoside(s) particularly, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target glycoside(s) particularly, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside O4 obtained according to this invention may be employed as a sweetening compound as the sole sweetener, or it may be used together with at least one naturally occurring high intensity sweeteners such as rebaudioside A, rebaudioside A2, rebaudioside A3, rebaudioside AM, rebaudioside B, rebaudioside C2, rebaudioside D, rebaudioside D2, rebaudioside D3, rebaudioside D4, rebaudioside E, rebaudioside E2, rebaudioside F, rebaudioside F2, rebaudioside F3, rebaudioside G, rebaudioside H, rebaudioside rebaudioside 12, rebaudioside 13, rebaudioside J, rebaudioside K2, rebaudioside L, rebaudioside M, rebaudioside M2, rebaudioside N, rebaudioside O, rebaudioside 02, rebaudioside O3, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside W, rebaudioside W2, rebaudioside W3, rebaudioside Y, rebaudioside Z1, rebaudioside Z2, steviolbioside, stevioside, stevioside D, stevioside E, stevioside E2, stevioside F, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside and combinations thereof.

In a particular embodiment, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 can be used in a sweetener composition comprising a compound selected from the group consisting of rebaudioside A, rebaudioside A2, rebaudioside A3, rebaudioside AM, rebaudioside B, rebaudioside C2, rebaudioside D, rebaudioside D2, rebaudioside D3, rebaudioside D4, rebaudioside E, rebaudioside E2, rebaudioside F, rebaudioside F2, rebaudioside F3, rebaudioside G, rebaudioside H, rebaudioside I, rebaudioside 12, rebaudioside 13, rebaudioside J, rebaudioside K2, rebaudioside L, rebaudioside M, rebaudioside M2, rebaudioside N, rebaudioside O, rebaudioside O2, rebaudioside O3, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside W, rebaudioside W2, rebaudioside W3, rebaudioside Y, rebaudioside Z1, rebaudioside Z2, steviolbioside, stevioside, stevioside D, stevioside E, stevioside E2, stevioside F, NSF-02, Mogroside V, Luo Han Guo, allulose, allose, D-tagatose, erythritol and combinations thereof.

Highly purified target glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 can be used in combination with natural sweetener suppressors such as gymnemic acid, hodulcin, ziziphin, lactisole, and others. Steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may also be combined with various umami taste enhancers. Steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 can be mixed with umami tasting and sweet amino acids such as glutamate, aspartic acid, glycine, alanine, threonine, proline, serine, glutamate, lysine, tryptophan and combinations thereof.

Highly purified target steviol glycoside(s) particularly, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Highly purified target steviol glycoside(s) particularly, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may be combined with reduced calorie sweeteners such as, for example, D-tagatose, L-sugars, L-sorbose, L-arabinose and combinations thereof.

Highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 obtained according to this invention may be applied as a foaming suppressor to produce zero calorie, reduced calorie or diabetic beverages and food products.

Examples of consumable products in which highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

Examples of consumable products in which highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 may be used as a flavor modifier or flavor with modifying properties include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 obtained in this invention may be used in dry or liquid forms.

The highly purified target steviol glycoside can be added before or after heat treatment of food products. The amount of the highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 depends on the purpose of usage. As discussed above, it can be added alone or in combination with other compounds.

The present invention is also directed to sweetness enhancement in beverages using steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 as a sweetness enhancer, wherein steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 is present in a concentration at or below their respective sweetness recognition thresholds.

As used herein, the term “sweetness enhancer” refers to a compound capable of enhancing or intensifying the perception of sweet taste in a composition, such as a beverage. The term “sweetness enhancer” is synonymous with the terms “sweet taste potentiator,” “sweetness potentiator,” “sweetness amplifier,” and “sweetness intensifier.”

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.5% to about 12% by weight, such as, for example, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.5% of about 10%, such as for example, from about 2% to about 8%, from about 3% to about 7% or from about 4% to about 6% by weight. In a particular embodiment, the sweetener is present in the beverage in an amount from about 0.5% to about 8% by weight. In another particular embodiment, the sweetener is present in the beverage in an amount from about 2% to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In yet another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a beverage comprising a sweetener comprising providing a beverage comprising a sweetener and adding a sweetness enhancer selected from steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 or a combination thereof, wherein steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 are present in a concentration at or below their sweetness recognition thresholds.

Addition of steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4 in a concentration at or below the sweetness recognition threshold to a beverage containing a sweetener may increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

The following examples illustrate preferred embodiments of the invention for the preparation of highly purified target steviol glycoside(s), particularly steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 and/or rebaudioside O4. It will be understood that the invention is not limited to the materials, proportions, conditions and procedures set forth in the examples, which are only illustrative.

### EXAMPLES

### Example 1

### Protein Sequences of Engineered Enzymes Used in the Biocatalytic Process

SEQ ID 1:
>SuSy_At, variant PM1-54-2-E05 (engineered
sucrose synthase; source of WT gene: Arabidopsis
thaliana)
MANAERMITRVHSQRERLNETLVSERNEVLALLSRVEAKGKGILQQNQII
AEFEALPEQTRKKLEGGPFFDLLKSTQEAIVLPPWVALAVRPRPGVWEYL
RVNLHALVVEELQPAEFLHFKEELVDGVKNGNFTLELDFEPFNASIPRPT
LHKYIGNGVDFLNRHLSAKLFHDKESLLPLLDFLRLHSHQGKNLMLSEKI
QNLNTLQHTLRKAEEYLAELKSETLYEEFEAKFEEIGLERGWGDNAERVL
DMIRLLLDLLEAPDPSTLETFLGRVPMVFNVVILSPHGYFAQDNVLGYPD
TGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVEL
SKELNGKPDLIIGNYSDGNLVASLLAHKLGVTQCTIAHALEKTKYPDSDI
YWKKLDDKYHFSCQFTADIFAMNHTDFIITSTFQEIAGSKETVGQYESHT
AFTLPGLYRVVHGIDVFDPKFNIVSPGADMSIYFPYTEEKRRLTKFHSEI
EELLYSDVENDEHLCVLKDKKKPILFTMARLDRVKNLSGLVEWYGKNTRL
RELVNLVVVGGDRRKESKDNEEKAEMKKMYDLIEEYKLNGQFRWISSQMD
RVRNGELYRYICDTKGAFVQPALYEAFGLTVVEAMTCGLPTFATCKGGPA
EIIVHGKSGFHIDPYHGDQAADLLADFFTKCKEDPSHWDEISKGGLQRIE
EKYTWQIYSQRLLTLTGVYGFWKHVSNLDRLEHRRYLEMFYALKYRPLAQ
AVPLAQDD
SEQ ID 2:
>UGTS12 variant 0234 (engineered
glucosyltransferase; source of WT gene: Solanum
lycopersicum)
MATNLRVLMFPWLAYGHISPFLNIAKQLADRGFLIYLCSTRINLESIIKK
IPEKYADSIHLIELQLPELPELPPHYHTTNGLPPHLNPTLHKALKMSKPN
FSRILQNLKPDLLIYDVLQPWAEHVANEQGIPAGKLLVSCAAVFSYFFSF
RKNPGVEFPFPAIHLPEVEKVKIREILAKEPEEGGRLDEGNKQMMLMCTS
RTIEAKYIDYCTELCNWKVVPVGPPFQDLITNDADNKELIDWLGTKPENS
TVFVSFGSEYFLSKEDMEEIAFALEASNVNFIWVVRFPKGEERNLEDALP
EGFLERIGERGRVLDKFAPQPRILNHPSTGGFISHCGWNSVMESIDFGVP
IIAMPIHNDQPINAKLMVELGVAVEIVRDDDGKIHRGEIAEALKSVVTGE
TGEILRAKVREISKNLKSIRDEEMDAVAEELIQLCRNSNKSK
SEQ ID 3:
>UGT76G1 variant 0042 (engineered
glucosyltransferase; source of WT gene: Stevia
rebaudiana)
MENKTETTVRRRRRIILFPVPFQGHINPILQLANVLYSKGFAITILHTNF
NKPKTSNYPHFTFRFILDNDPQDERISNLPTHGPLAGMRIPIINEHGADE
LRRELELLMLASEEDEEVSCLITDALWYFAQDVADSLNLRRLVLMTSSLF
NFHAHVSLPQFDELGYLDPDDKTRLEEQASGFPMLKVKDIKSAYSNWQIG
KEILGKMIKQTKASSGVIWNSFKELEESELETVIREIPAPSFLIPLPKHL
TASSSSLLDHDRTVFEWLDQQAPSSVLYVSFGSTSEVDEKDFLEIARGLV
DSGQSFLWVVRPGFVKGSTWVEPLPDGFLGERGKIVKWVPQQEVLAHPAI
GAFWTHSGWNSTLESVCEGVPMIFSSFGGDQPLNARYMSDVLRVGVYLEN
GWERGEVVNAIRRVMVDEEGEYIRQNARVLKQKADVSLMKGGSSYESLES
LVSYISSL
SEQ ID 4:
>UDP-glycosyltransferase 91C1 (UG1yT91C1;
source of WT gene: Oryza sativa Japonica;
NCBI Reference Sequence: XP_015629141.1)
MDSGYSSSYAAAAGMHVVICPWLAFGHLLPCLDLAQRLASRGHRVSFVST
PRNISRLPPVRPALAPLVAFVALPLPRVEGLPDGAESTNDVPHDRPDMVE
LHRRAFDGLAAPFSEFLGTACADWVIVDVFHHWAAAAALEHKVPCAMMLL
GSAHMIASIADRRLERAETESPAAAGQGRPAAAPTFEVARMKLIRTKGSS
GMSLAERFSLTLSRSSLVVGRSCVEFEPETVPLLSTLRGKPITFLGLMPP
LHEGRREDGEDATVRWLDAQPAKSVVYVALGSEVPLGVEKVHELALGLEL
AGTRFLWALRKPTGVSDADLLPAGFEERTRGRGVVATRWVPQMSILAHAA
VGAFLTHCGWNSTIEGLMFGHPLIMLPIFGDQGPNARLIEAKNAGLQVAR
NDGDGSFDREGVAAAIRAVAVEEESSKVFQAKAKKLQEIVADMACHERYI
DGFIQQLRSYKD

### Example 2

Expression and formulation of SuSy_At variant of SEQ ID 1

The gene coding for the SuSy_At variant of SEQ ID 1 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.2 mM) and carried out at 30° C. and 200 rpm for 16-18 hours.

Cells were harvested by centrifugation (3220×g, 20 min, 4° C.) and re-suspended to an optical density of 200 (measured at 600 nm (0D600)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at to, 3 mM MgCl₂, and 15 mM uridine diphosphate (UDP).

### Example 3

### Expression and Formulation of UGTSl2 Variant of SEQ ID 2

The gene coding for the UGTSl2 variant of SEQ ID 2 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTSl2, activity in Units is defined as follows: 1 mU of UGTSl2 turns over 1 nmol of rebaudioside A (Reb A) into rebaudioside D (Reb D) in 1 minute. Reaction conditions for the assay are 30° C., 50 mM potassium phosphate buffer pH 7.0, 10 mM Reb A at to, 500 mM sucrose, 3 mM MgCl₂, 0.25 mM uridine diphosphate (UDP) and 3 U/mL of SuSy_At.

### Example 4

### Expression and Formulation of UGT76G1 Variant of SEQ ID 3

The gene coding for the UGT76G1 variant of SEQ ID 3 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (0D600)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M (Reb M) in 1 minute. Reaction conditions for the assay are 30° C., 50 mM potassium phosphate buffer pH 7.0, 10 mM Reb D at to, 500 mM sucrose, 3 mM MgCl₂, 0.25 mM uridine diphosphate (UDP) and 3 U/mL of SuSy_At.

### Example 5

Synthesis of Rebaudioside N2 and Rebaudioside O4 from Rebaudioside C in a One-Pot Reaction, Adding UGTSl2, SuSy_At and UGT76G1 at the Same Time.

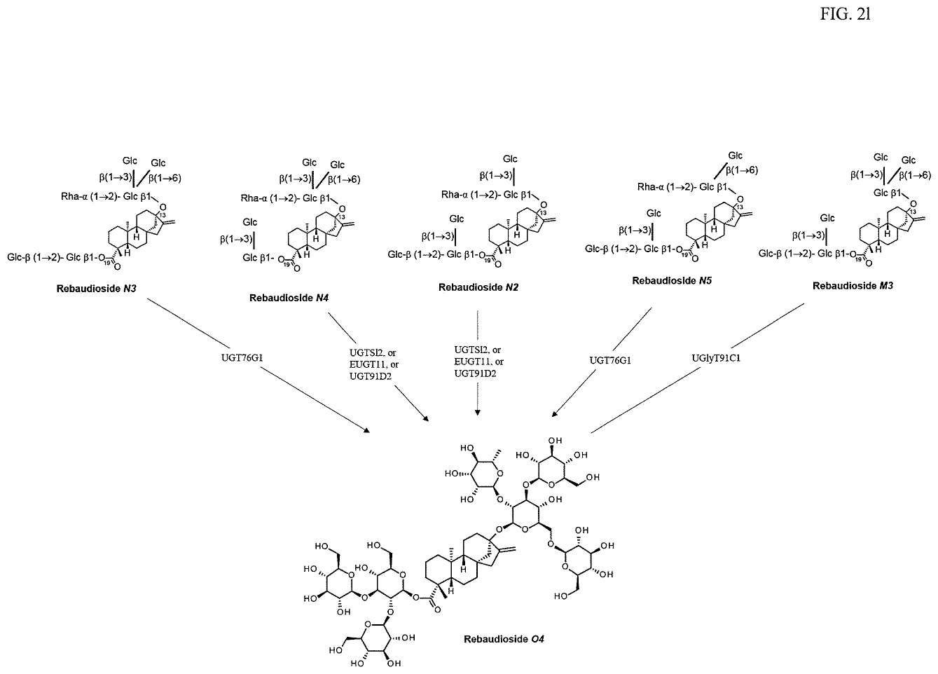

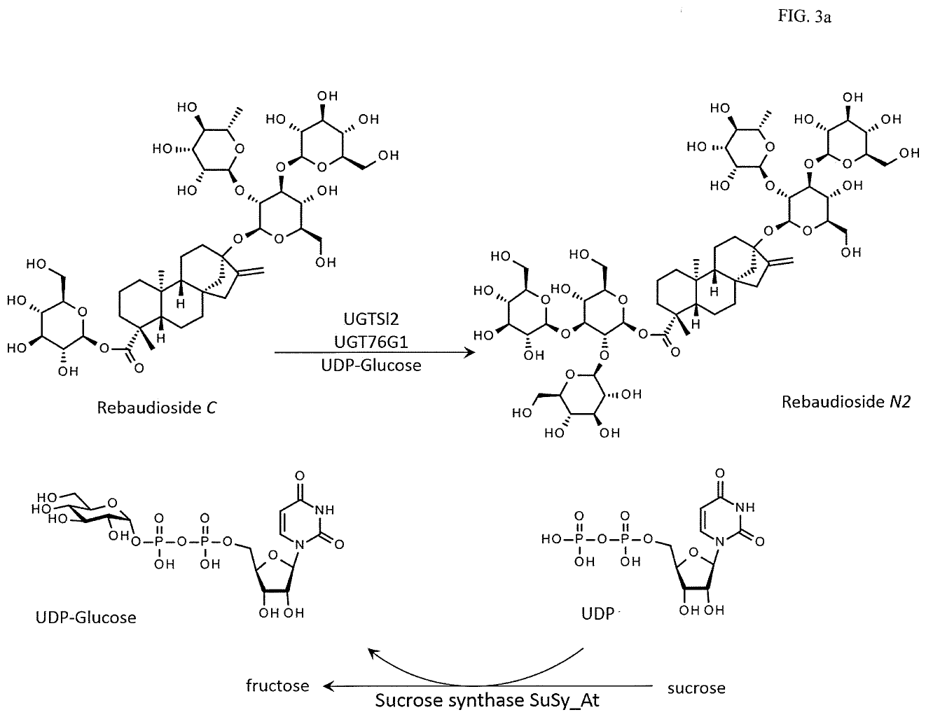

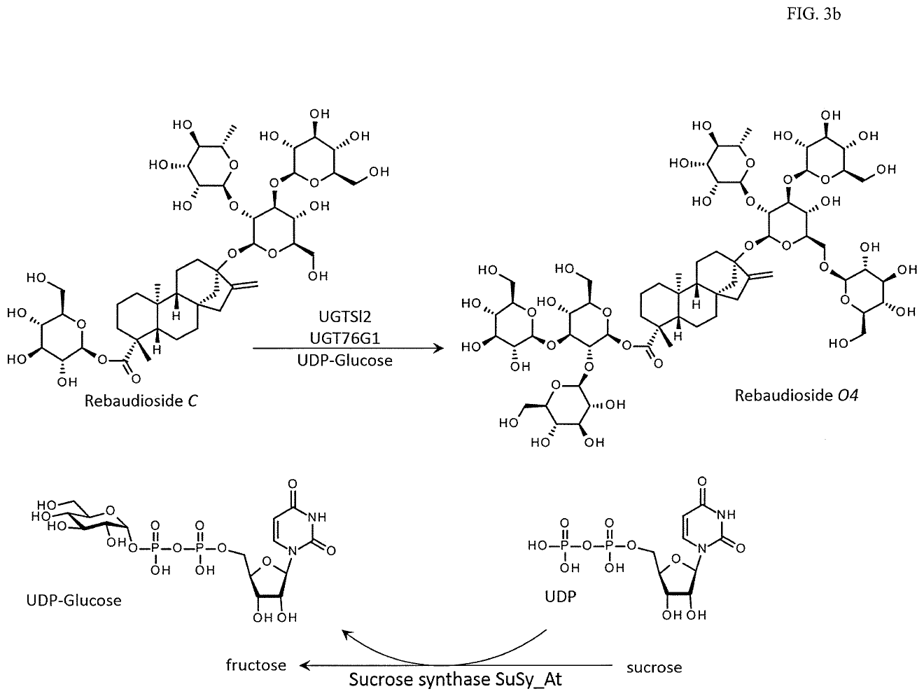

Rebaudioside N2 (reb N2) and rebaudioside O4 (reb O4) were synthesized directly from rebaudioside C in a one-pot reaction (see FIG. 3a and FIG. 3b), utilizing the three enzymes (see EXAMPLES 1, 2, 3 and 4): UGTSl2 (variant of SEQ ID 2), SuSy_At (variant of SEQ ID 1) and UGT76G1 (variant of SEQ ID 3). The final reaction solution contained 105 U/L UGTSl2, 405 U/L SuSy_At, 3 U/L UGT76G1, 5 mM rebaudioside C, 0.25 mM uridine diphosphate (UDP), 1 M sucrose, 4 mM MgCl₂ and potassium phosphate buffer (pH 6.6). First, 207 mL of distilled water were mixed with 0.24 g MgCl₂.6H₂O, 103 g sucrose, 9.9 mL of 1.5 M potassium phosphate buffer (pH 6.6) and 15 g rebaudioside C. After dissolving the components, the temperature was adjusted to 45° C. and UGTSl2, SuSy_At, UGT76G1 and 39 mg UDP were added. The reaction mixture was incubated at 45° C. shaker for 24 hrs. Additional 39 mg UDP was added at 8 hrs and 18 hours. The content of reb O4, reb N2, reb K, and reb C at several time points was analyzed by HPLC.

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB—C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

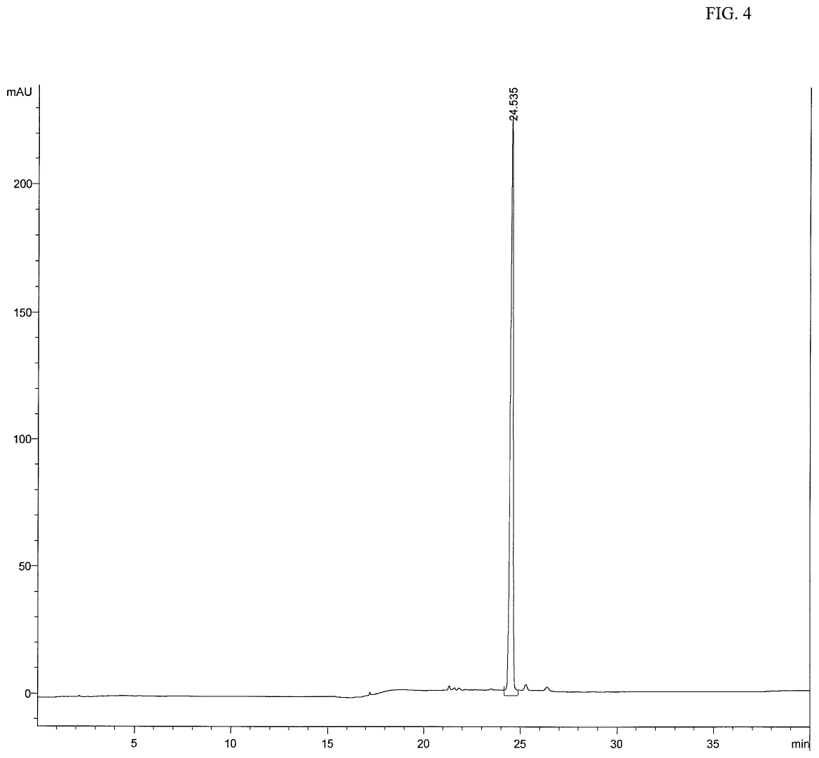

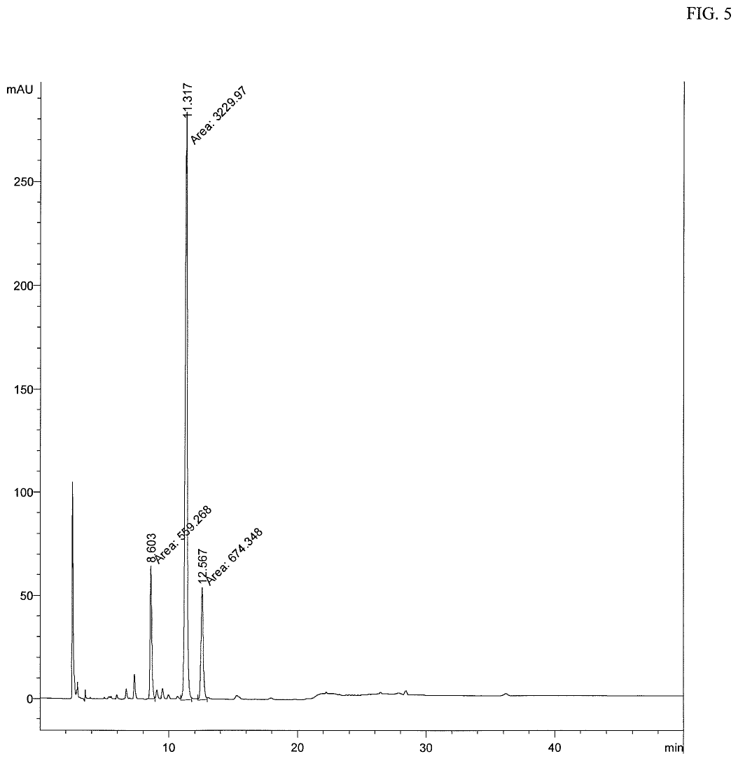

Table 3 shows for each time point the conversion of Reb C into identified rebaudioside species (area percentage). The chromatograms of rebaudioside C and the reaction mixture at 96 hours are shown in FIG. 4 and FIG. 5 respectively. Those with skill in the art will appreciate that retention times can occasionally vary with changes in solvent and/or equipment.

TABLE 3
Biotransformation of reb C to reb N2 and reb O4

% conversion from reb C

| Time, hrs | Reb O4 | Reb N2 | Reb K | Reb C |
|---|---|---|---|---|
| 0 | 0 | 0 | 0 | 100 |
| 24 | 4.4 | 34.2 | 31.7 | 29.6 |
| 96 | 12.5 | 72.4 | 15.1 | 0 |

### Example 6

### Purification of Rebaudioside N2 and O4

40 mL of the reaction mixture of EXAMPLE 5, (after 96 hrs), was inactivated by adjusting the pH to pH 5.5 with H₃PO₄ and then boiled for 10 minutes and filtered. The filtrate was loaded into a column containing 30 mL YWD03 (Cangzhou Yuanwei, China) resin pre-equilibrated with water. The resin was washed with 150 mL warm water (35° C.) and the water effluent from this step was discarded.

The steviol glycosides were eluted from the YWD03 resin column by elution with 200 mL 70% v/v ethanol/water. The effluent from this step was collected and subjected to evaporation on a rotary evaporator until the volume of the sample was approximately 25 mL. This concentrated sample was subjected to further purification by preparative HPLC, using the conditions listed in Table 4 below.

Preparative HPLC fractions that corresponded to individual compounds from multiple runs were combined according to retention time. The samples were freeze-dried to give 60 mg rebaudioside N2 and 10 mg rebaudioside O4.

TABLE 4
Conditions for Preparative HPLC

| Column | Agilent Poroshell 120 SB-C18, | | |
|---|---|---|---|
| | 4.6 mm × 150 mm, 2.7 μm | | |
| Temperature | 40° C. | | |
| Mobile Phase | B - Water | | |
| | C - Acetonitrile | | |
| Gradient | Time (min) | C | B |
| | 0 | 25 | 75 |
| | 10.0 | 25 | 75 |
| | 13.0 | 50 | 50 |
| | 17.0 | 50 | 50 |

| | |
|---|---|
| Flow rate | 1 mL/min |
| Injection | 10 μL |
| Stop time | 22 mins |
| Post time | 10 mins |
| Autosampler | Ambient |
| temperature | |
| Detection | UV at 210 nm |

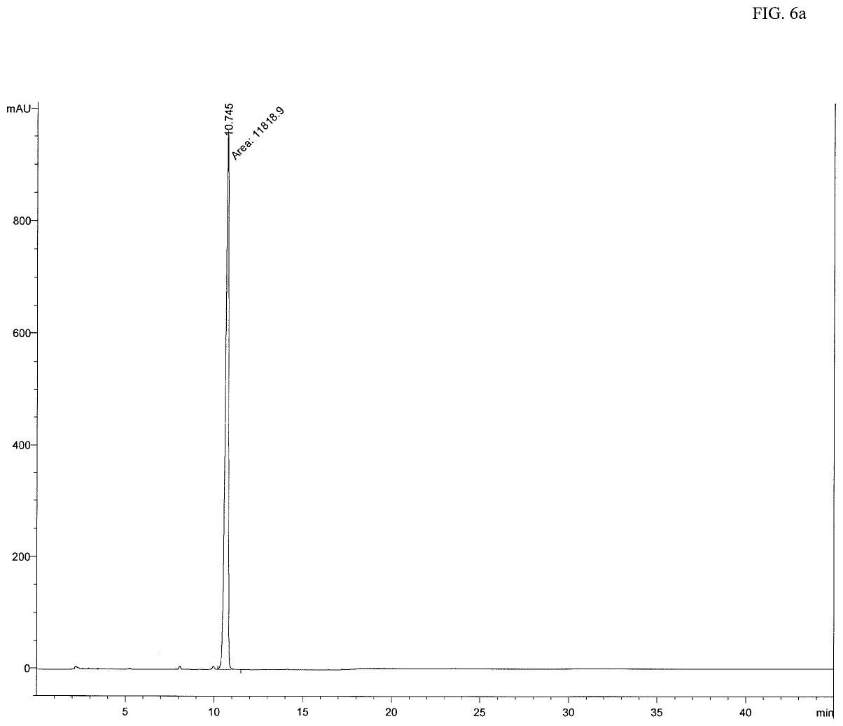

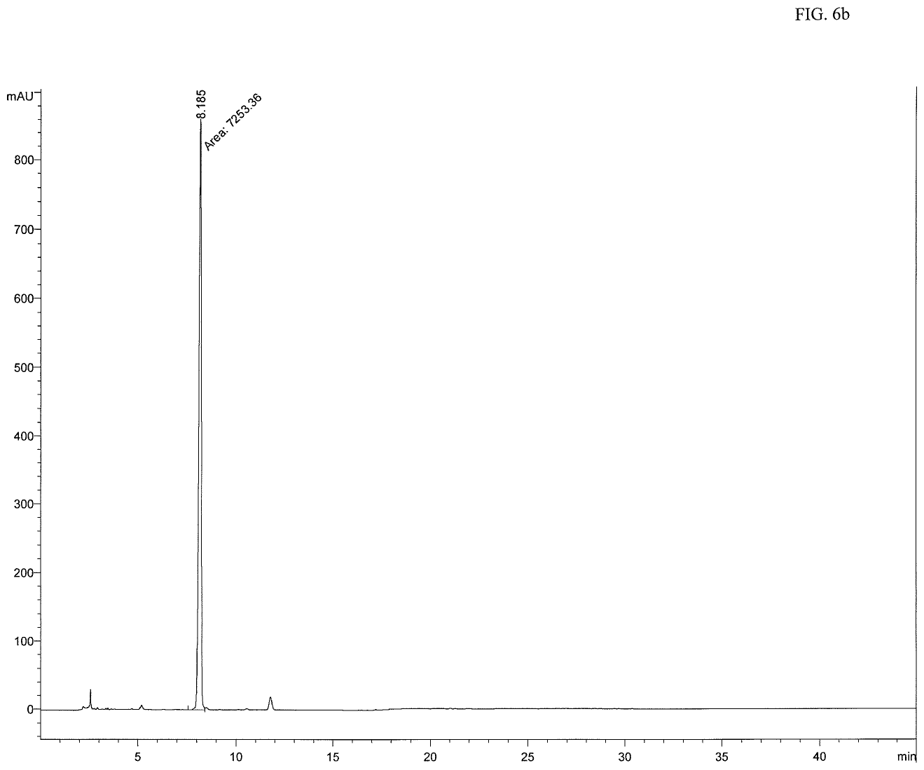

The chromatograms of purified reb N2 and reb O4 are shown in FIGS. 6a and 6b, respectively. Those with skill in the art will appreciate that retention times can occasionally vary with changes in solvent and/or equipment.

### Example 7

### Structure Elucidation of Rebaudioside N2

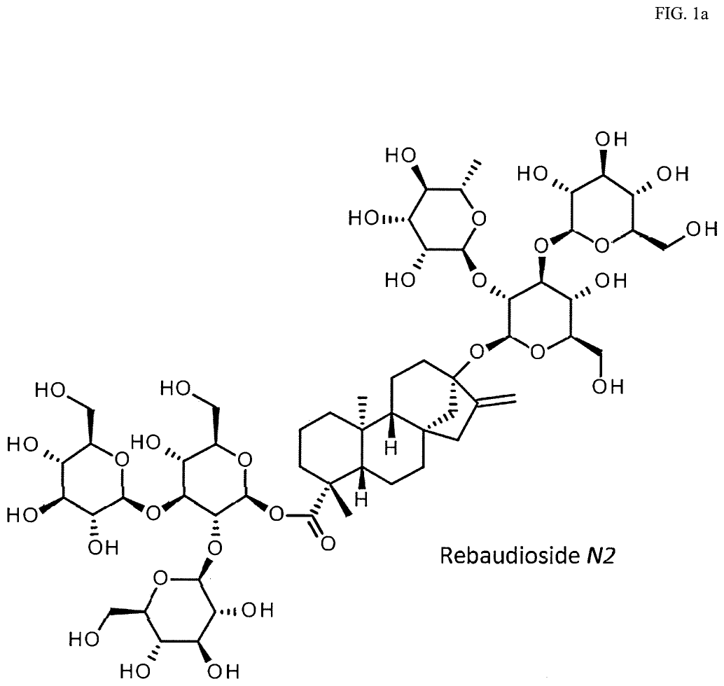

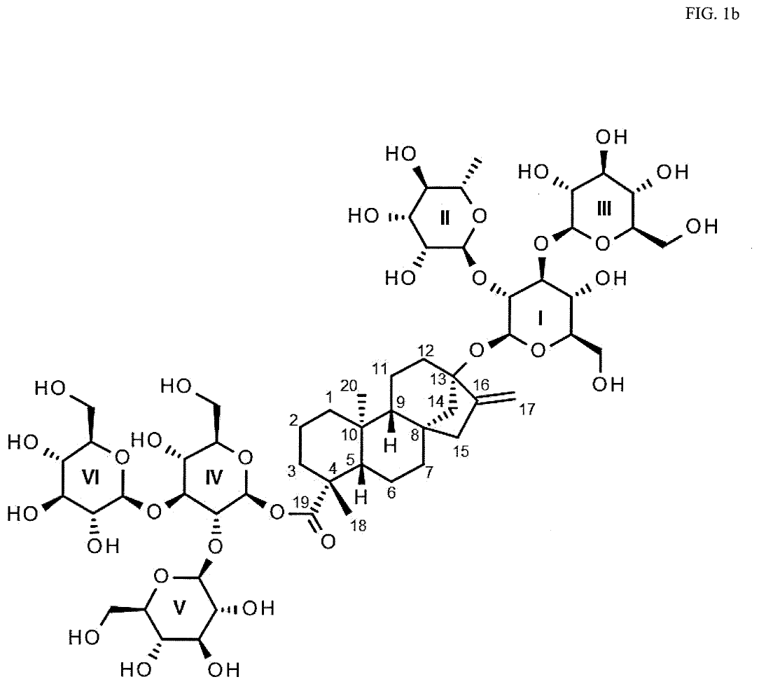

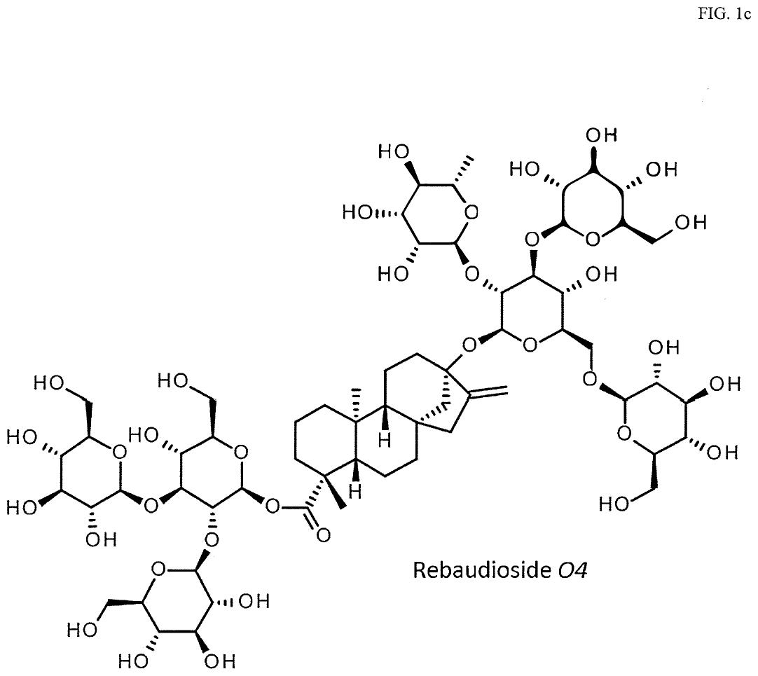

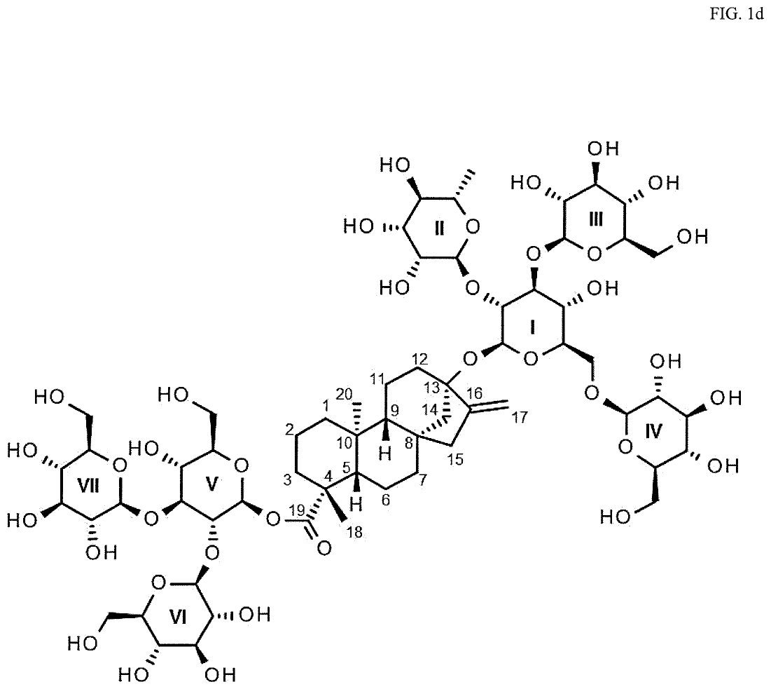

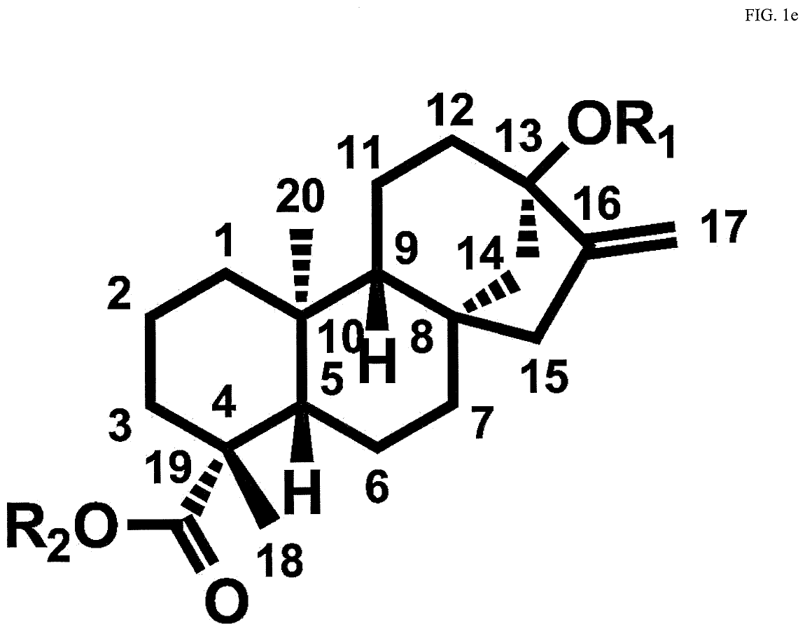

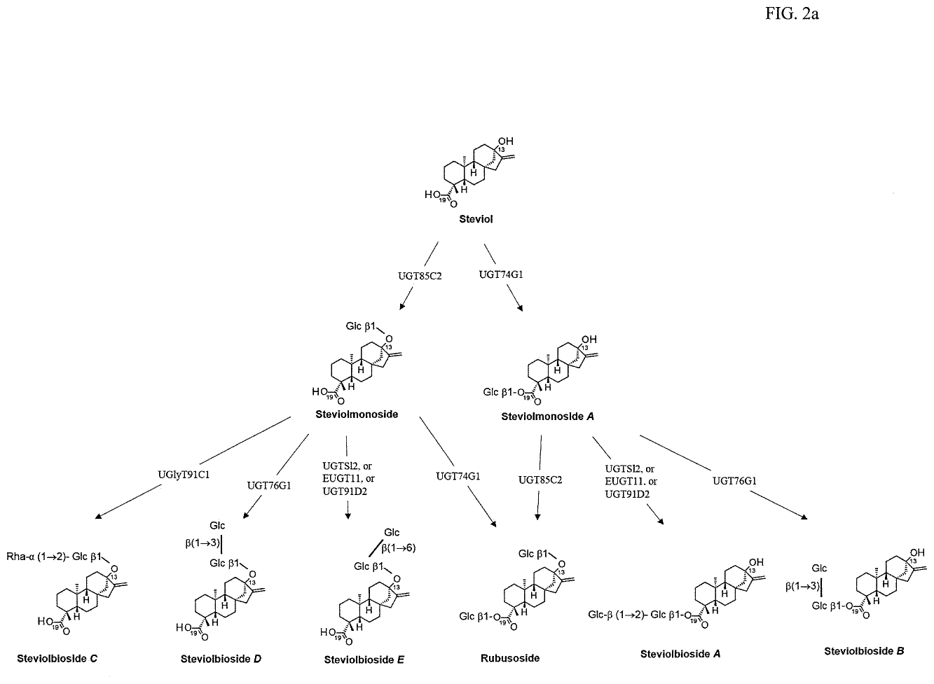

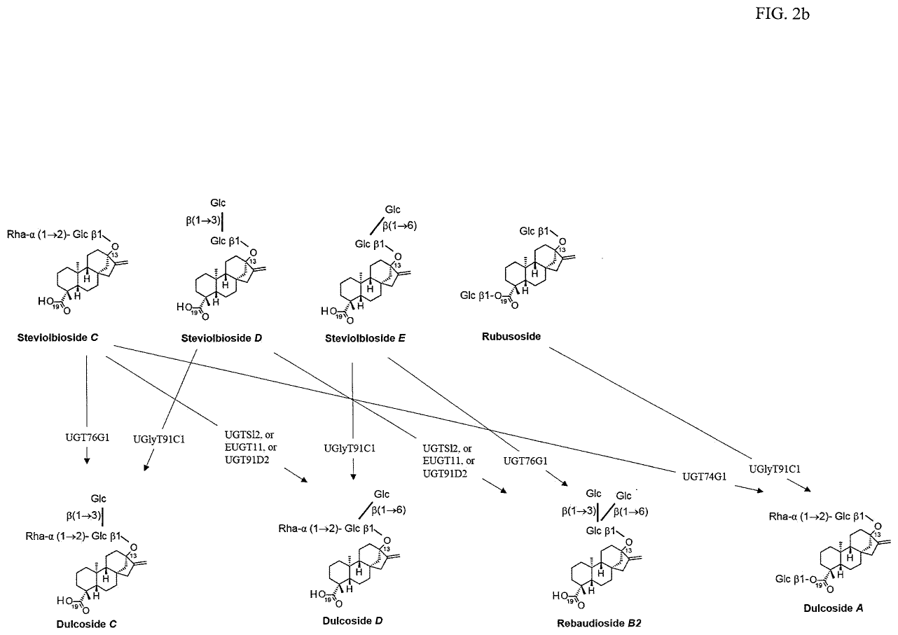

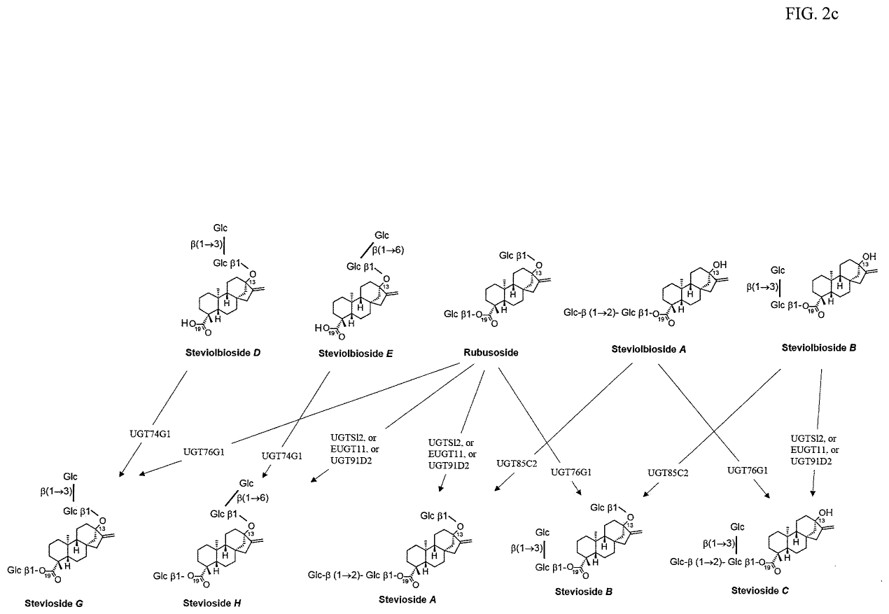

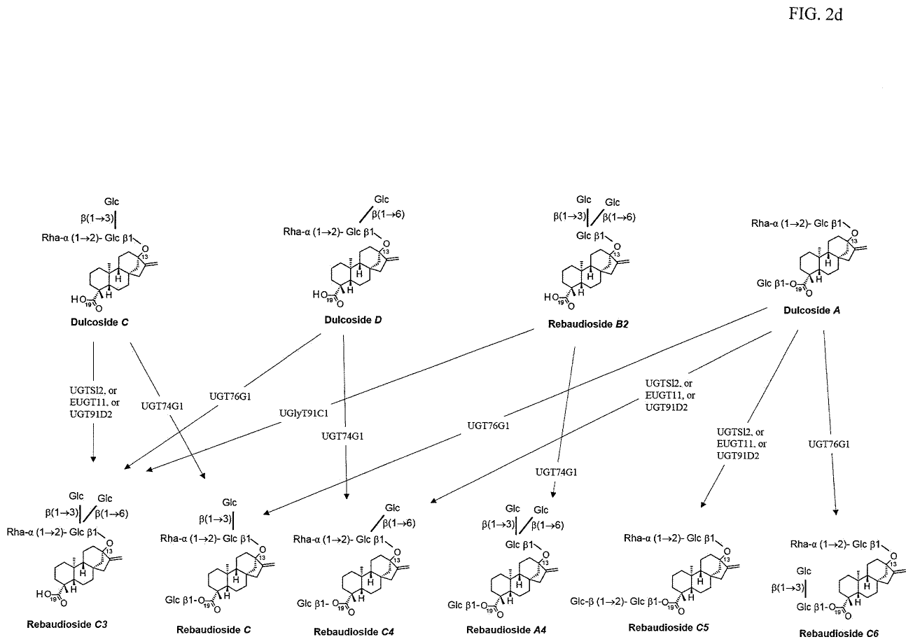

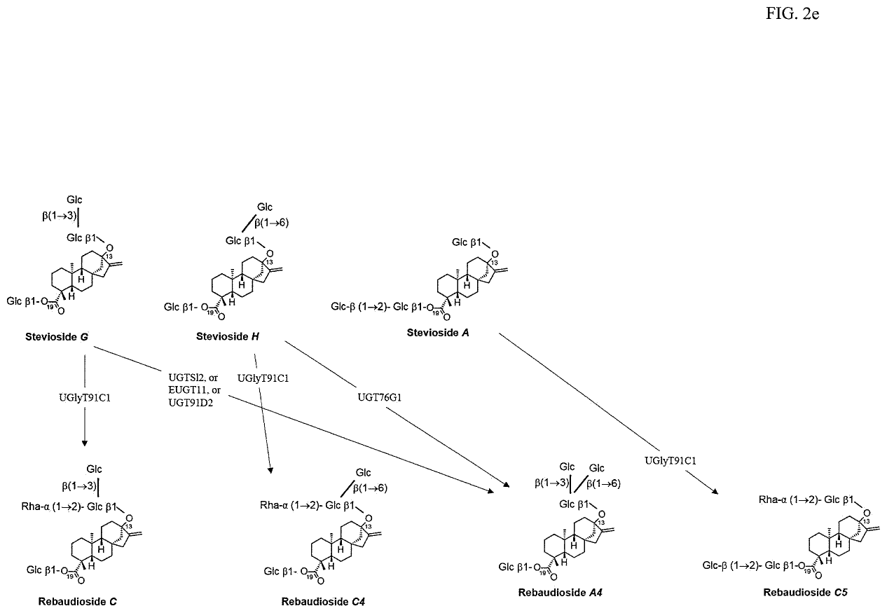

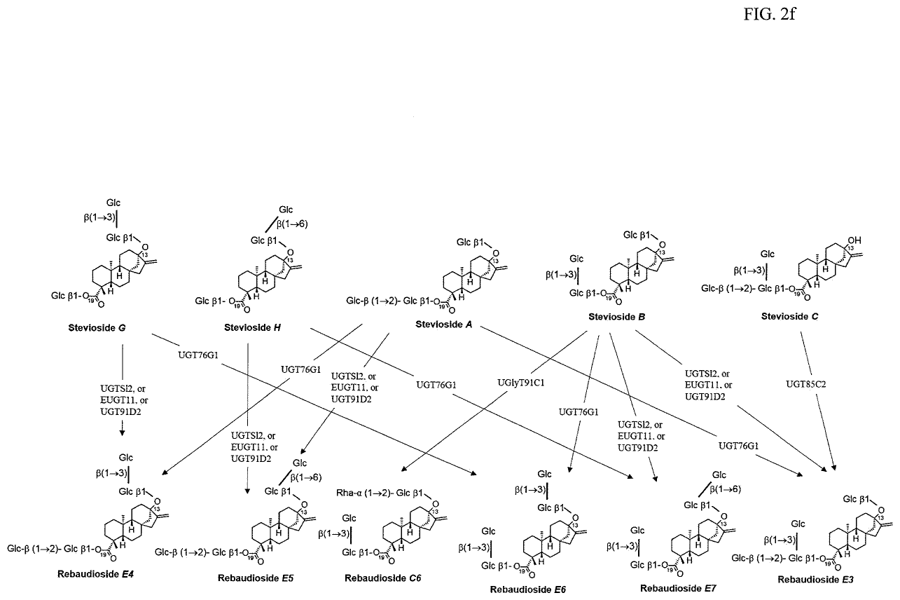

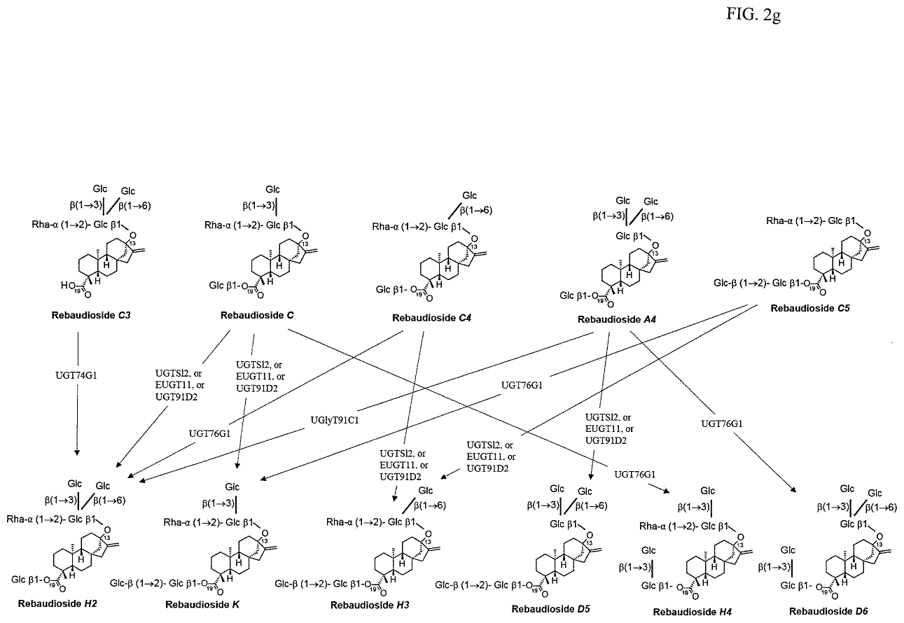

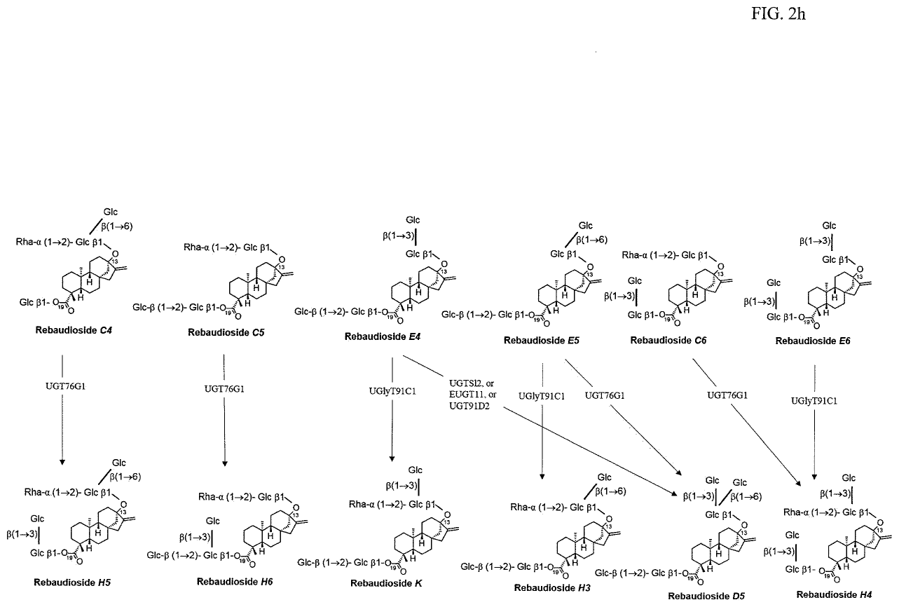

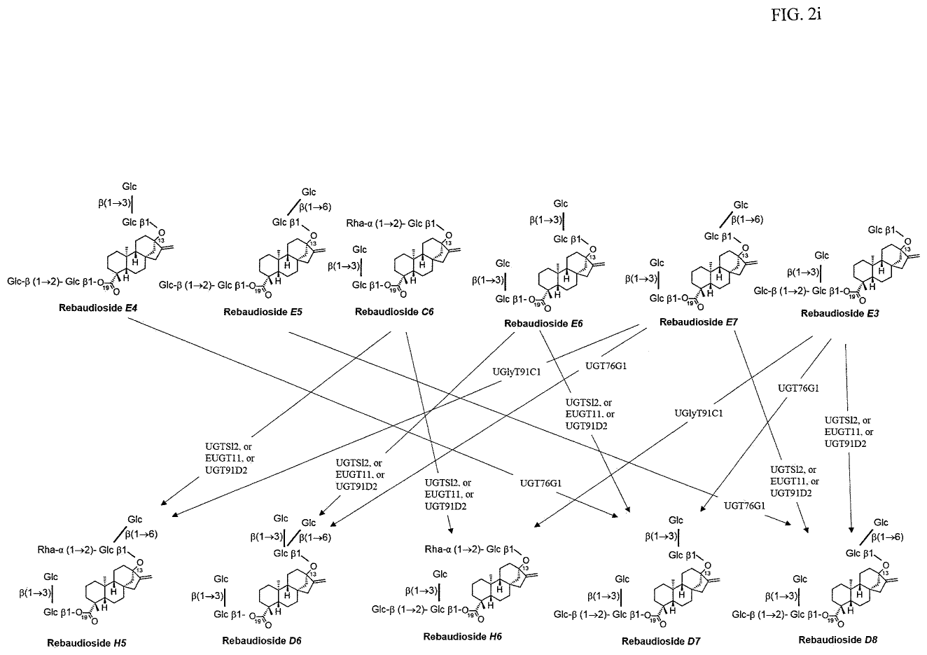

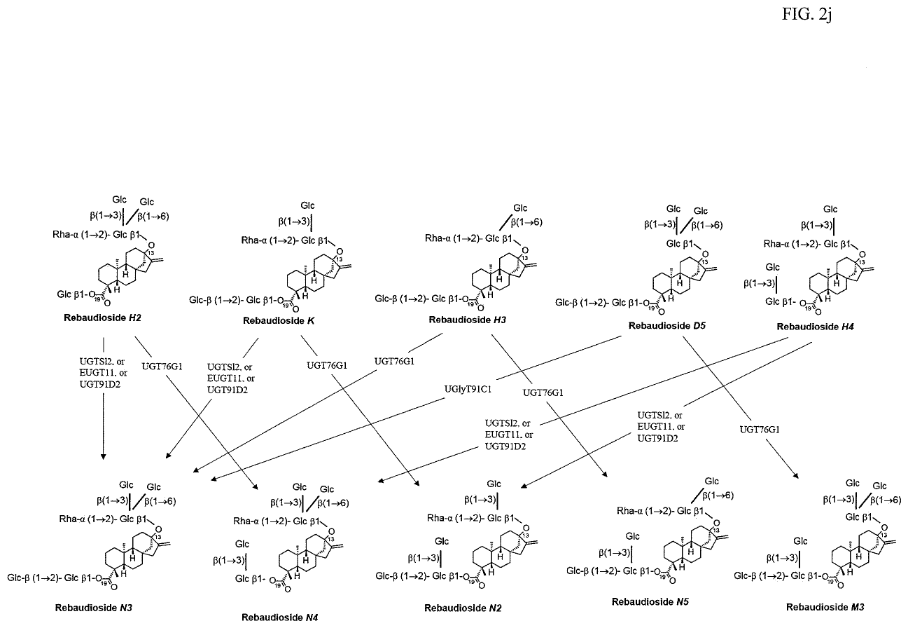

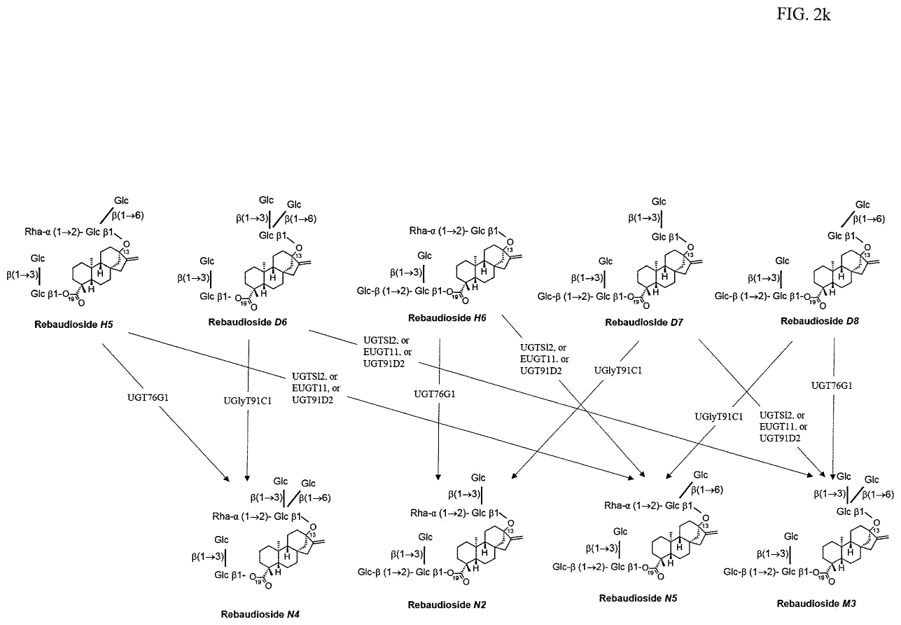

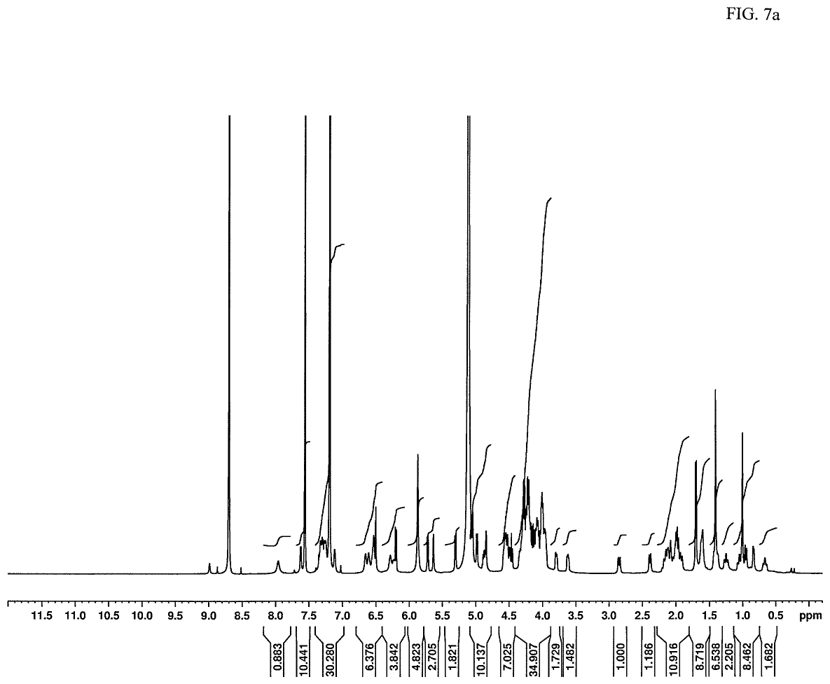

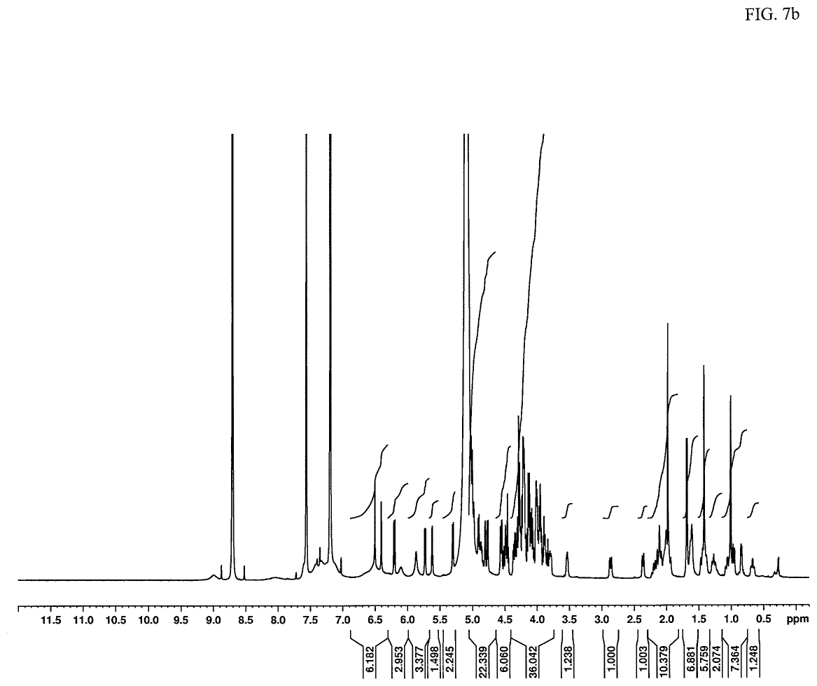

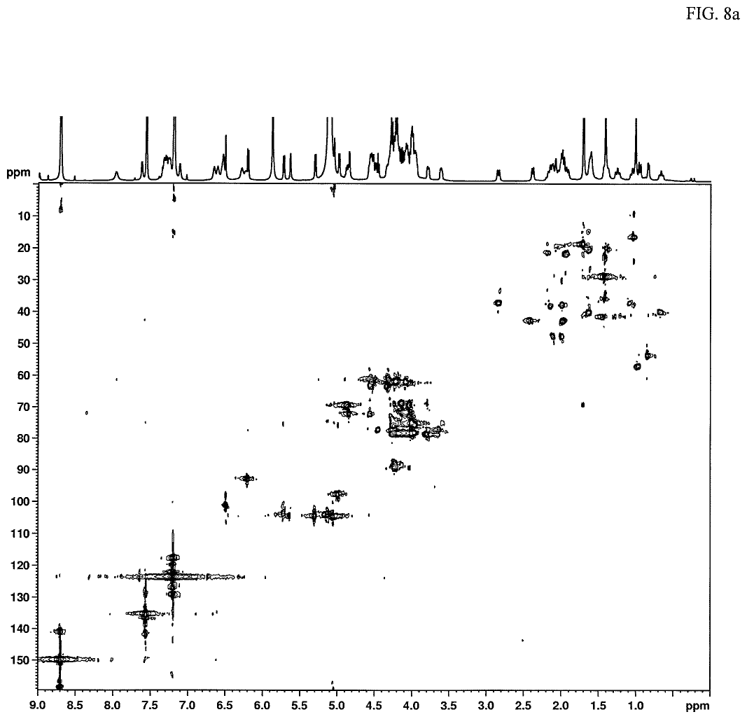

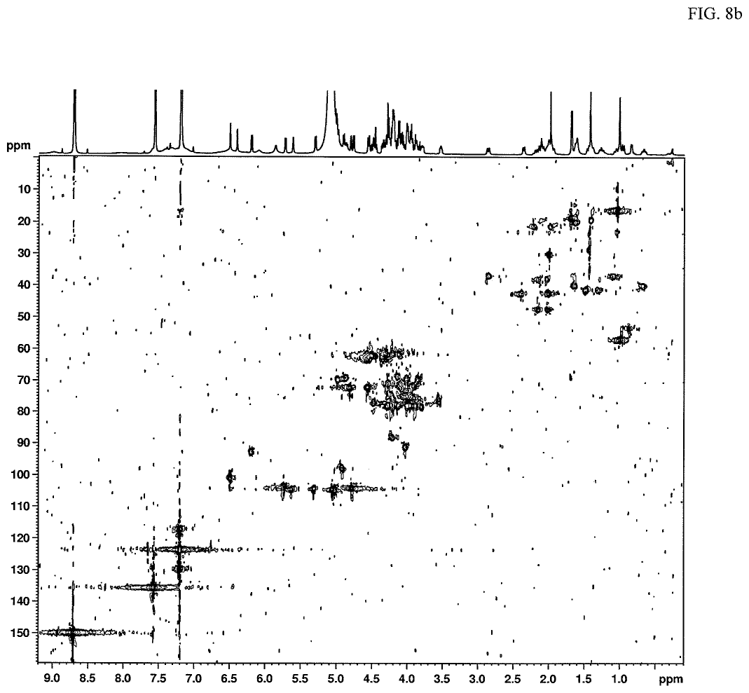

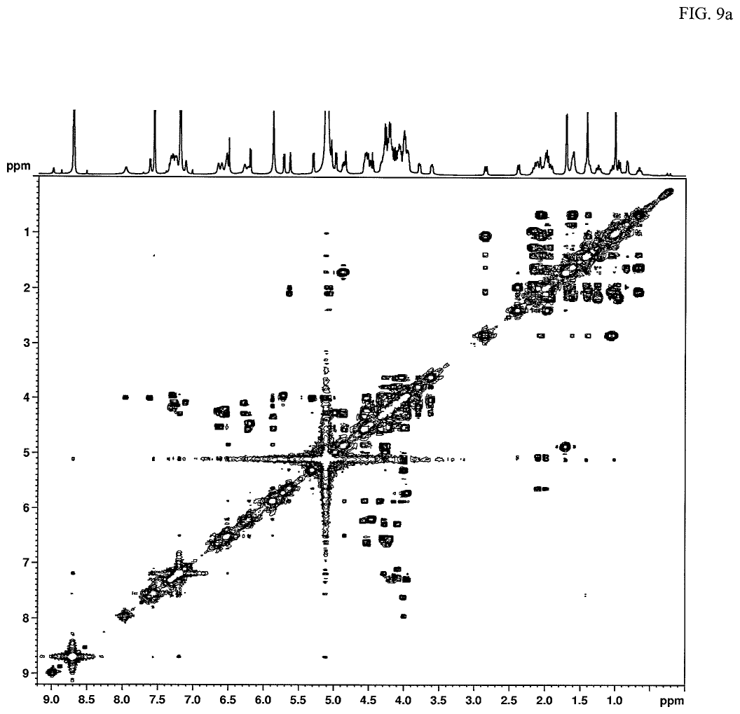

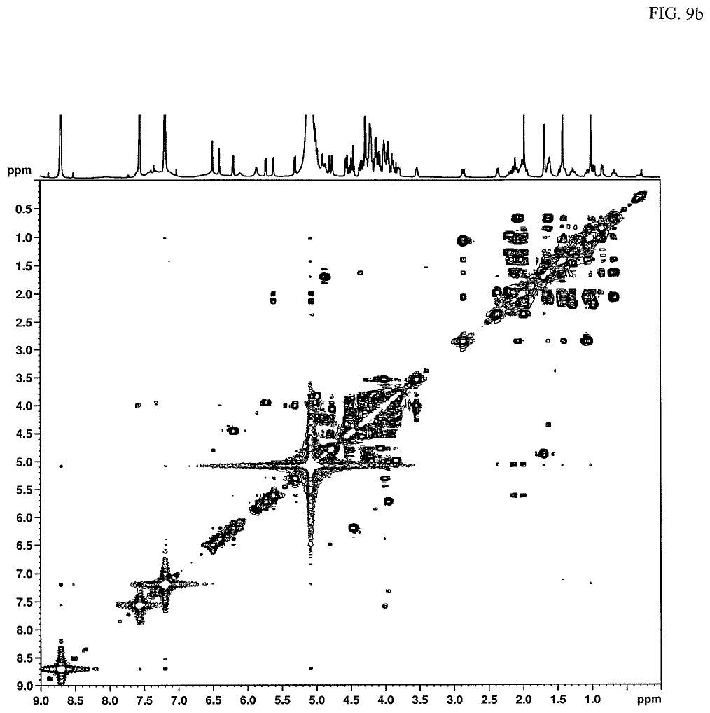

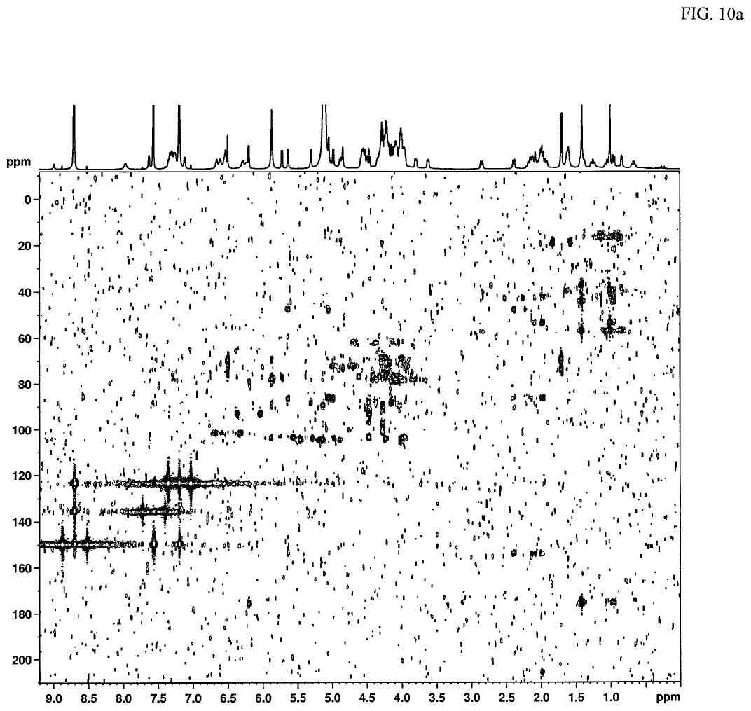

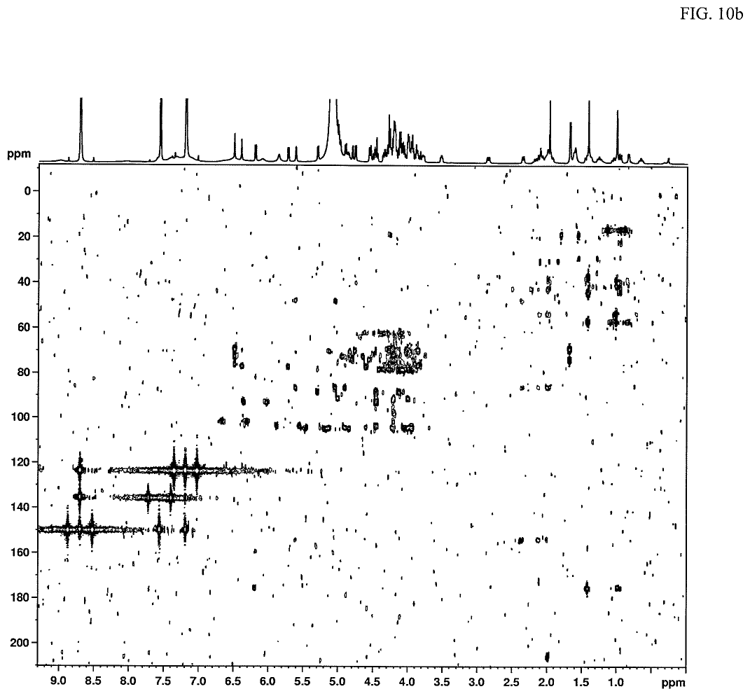

NMR experiments were performed on a Bruker 500 MHz spectrometer, with the sample dissolved in pyridine-d5. Along with signals from the sample, signals from pyridine-d5 at δ_{C} 123.5, 135.5, 149.9 ppm and ox 7.19, 7.55, 8.71 ppm were observed. ¹H-NMR spectrum of rebaudioside N2 recorded in pyridine-d5 confirmed the excellent quality of the sample (see FIG. 7a). HSQC (see FIG. 8a) shows the presence of an exo-methylene group in the sugar region with a long-range coupling to C-15, observable in the H,H—COSY (FIG. 9a). Other deep-fielded signals of the quaternary carbons (C-13, C-16 and C-19) are detected by the HMBC (FIG. 10a). Correlation of the signals in the HSQC, HMBC and H,H—COSY reveal the presence of steviol glycoside with the following aglycone structure (see FIG. 1e):

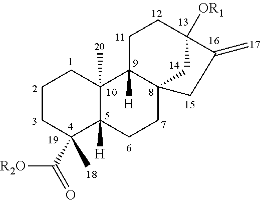

HSQC spectrum of rebaudioside N2 also shows the presence of a methyl-doublet (J=6.3 Hz) with a H,H—COSY correlation to a signal in the sugar region. HSQC also shows the presence of six anomeric signals, of which five of these anomeric signals have coupling constants of about 8 Hz and the broad signals of their sugar linkage allow the identification of these six sugars as β-D-glucosides. The sixth anomeric signal is a broad singlet and the H,H—COSY correlation to the methyl doublet allow to the identification this sugar as α-L-rhamnose.

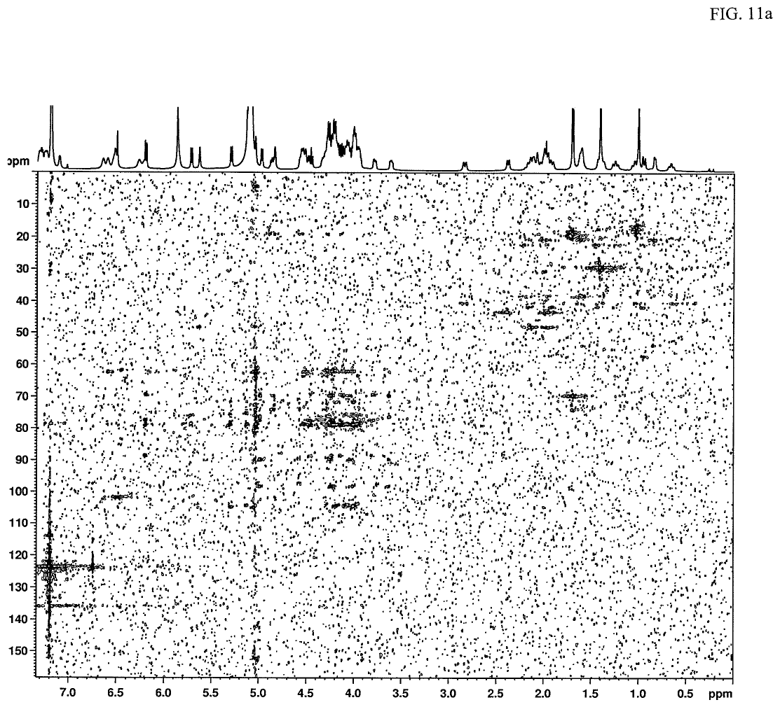

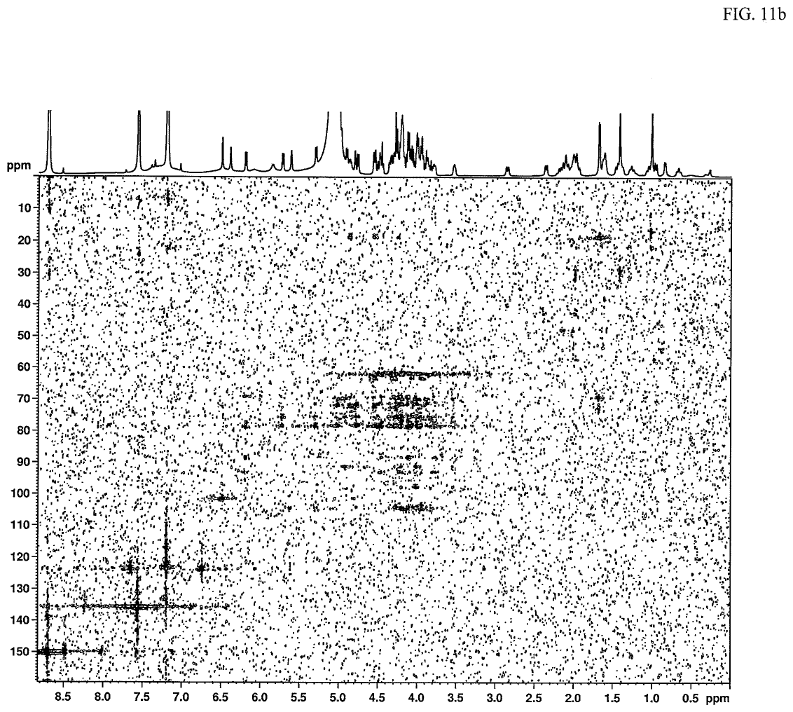

The observation of the anomeric protons in combination with HSQC and HMBC reveal the sugar linkage and the correlation to the aglycone. The assignment of the sugar sequence was confirmed by using the combination of HSQC-TOCSY (FIG. 11a) and HSQC.

Altogether, results from NMR experiments above were used to assign the chemical shifts of the protons and carbons of the structure of rebaudioside N2 (see Table 5).

TABLE 5
Chemical shifts of rebaudioside N2

| | | | | |
|---|---|---|---|---|
| Position | δ_{C} [ppm] | δ_{H} [ppm] | J [Hz]/(INT) | HMBC (H −> C) |

Aglycone moiety

| 1 | 39.9 | t | 0.67 | m | | |
|---|---|---|---|---|---|---|
| | | | 1.62 | m | | |
| 2 | 19.1 | t | 1.41 | m | | |
| | | | 2.06 | m | | |
| 3 | 37.0 | t | 1.07 | m | | |
| | | | 2.84 | m | | |
| 4 | 44.1 | s | — | | | |
| 5 | 56.9 | d | 0.96 | m | | |
| 6 | 21.4 | t | 1.92 | m | | |
| | | | 2.17 | m | | |
| 7 | 41.2 | t | 1.25 | m | | |
| | | | 1.44 | m | | |
| 8 | 42.8 | s | — | | | |
| 9 | 53.8 | d | 0.84 | m | | |
| 10 | 39.0 | s | — | | | |
| 11 | 20.2 | t | 1.60 | m | | |
| | | | 1.63 | m | | |
| 12 | 37.9 | t | 1.98 | m | | |
| | | | 2.13 | m | | |
| 13 | 86.2 | s | — | | | |
| 14 | 42.8 | t | 1.95 | d | 11.0 | |
| | | | 2.39 | d | 11.0 | |
| 15 | 47.6 | t | 1.99 | d | 15.8 | 7, 8, 9, 14 |
| | | | 2.10 | d | 15.8 | |
| 16 | 154.0 | s | — | | | |
| 17 | 104.4 | t | 5.04 | br s | | 13, 15, 16 |
| | | | 5.63 | br s | | |
| 18 | 28.7 | q | 1.41 | s | (3H) | 3, 4, 5, 19 |
| 19 | 175.4 | s | — | | | |
| 20 | 16.2 | q | 1.01 | s | (3H) | 1, 5, 9, 10 |

Sugar moiety
Sugar I: β-D-Glucopyranoside

| | | | | | | |
|---|---|---|---|---|---|---|
| 1ⁱ | 97.7 | d | 4.99 | d | 7.7 | 13 |
| 2ⁱ | 75.3 | d | 4.27 | m | | |
| 3ⁱ | 89.1 | d | 4.23 | m | | |
| 4ⁱ | 69.1 | d | 4.03 | m | | |
| 5ⁱ | 77.0 | d | 3.63 | m | | |
| 6ⁱ | 61.6 | t | 4.10 | m | | |
| | | | 4.27 | m | | |

Sugar II: α-L-Rhamnopyranoside

| | | | | | | |
|---|---|---|---|---|---|---|
| 1ⁱⁱ | 101.1 | d | 6.50 | br s | | 2ⁱ |
| 2ⁱⁱ | 71.8 | d | 4.84 | m | | |
| 3ⁱⁱ | 72.0 | d | 4.56 | m | | |
| 4ⁱⁱ | 73.3 | d | 4.26 | m | | |
| 5ⁱⁱ | 69.0 | d | 4.87 | m | | |
| 6ⁱⁱ | 18.4 | q | 1.71 | d | 6.3 | |

Sugar III: β-D-Glucopyranoside

| | | | | | | |
|---|---|---|---|---|---|---|
| 1ⁱⁱⁱ | 104.0 | d | 5.13 | d | 8.1 | 3ⁱ |
| 2ⁱⁱⁱ | 74.8 | d | 4.00 | m | | |
| 3ⁱⁱⁱ | 78.0 | d | 4.14 | m | | |
| 4ⁱⁱⁱ | 71.3 | d | 4.05 | m | | |
| 5ⁱⁱⁱ | 78.0 | d | 3.98 | m | | |
| 6ⁱⁱⁱ | 62.0 | t | 4.20 | m | | |
| | | | 4.54 | m | | |

Sugar IV: β-D-Glucopyranoside

| | | | | | | |
|---|---|---|---|---|---|---|
| 1^{iv} | 92.6 | d | 6.21 | d | 7.7 | 19 |
| 2^{iv} | 77.0 | d | 4.47 | m | | |
| 3^{iv} | 87.7 | d | 4.22 | m | | |
| 4^{iv} | 68.6 | d | 4.13 | m | | |
| 5^{iv} | 78.3 | d | 3.80 | m | | |
| 6^{iv} | 61.7 | t | 4.03 | m | | |
| | | | 4.23 | m | | |

Sugar V: β-D-Glucopyranoside

| | | | | | | |
|---|---|---|---|---|---|---|
| 1^{v} | 103.4 | d | 5.72 | d | 8.1 | 2^{iv} |
| 2^{v} | 75.4 | d | 3.98 | m | | |
| 3^{v} | 78.0 | d | 4.07 | m | | |
| 4^{v} | 71.0 | d | 4.10 | m | | |
| 5^{v} | 78.3 | d | 4.00 | m | | |
| 6^{v} | 61.5 | t | 4.19 | m | | |
| | | | 4.53 | m | | |

Sugar VI: β-D-Glucopyranoside

| | | | | | | |
|---|---|---|---|---|---|---|
| 1^{vi} | 104.3 | d | 5.30 | d | 7.7 | 3^{iv} |
| 2^{vi} | 75.2 | d | 4.00 | m | | |
| 3^{vi} | 78.1 | d | 4.17 | m | | |
| 4^{vi} | 71.3 | d | 4.09 | m | | |
| 5^{vi} | 77.7 | d | 3.97 | m | | |
| 6^{vi} | 61.7 | t | 4.20 | m | | |
| | | | 4.28 | m | | |

Correlation of all NMR results indicates rebaudioside N2 with five β-D-glucoses and one α-L-rhamnose attached to steviol aglycone, as depicted with the following chemical structure (see FIGS. 1a and 1b):

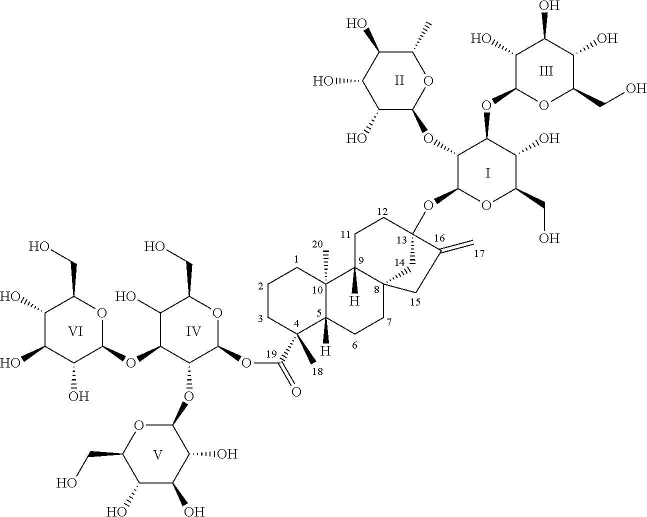

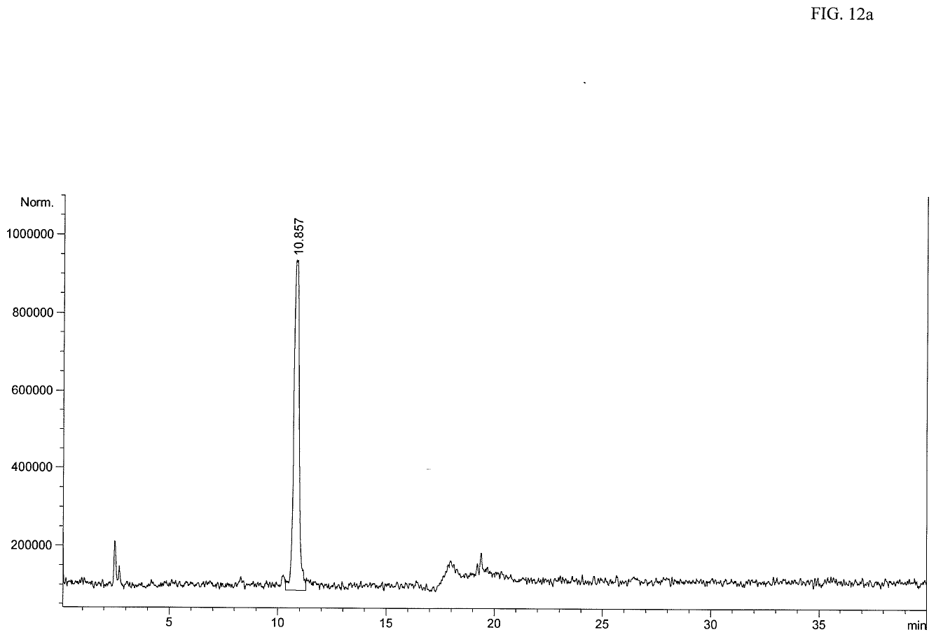

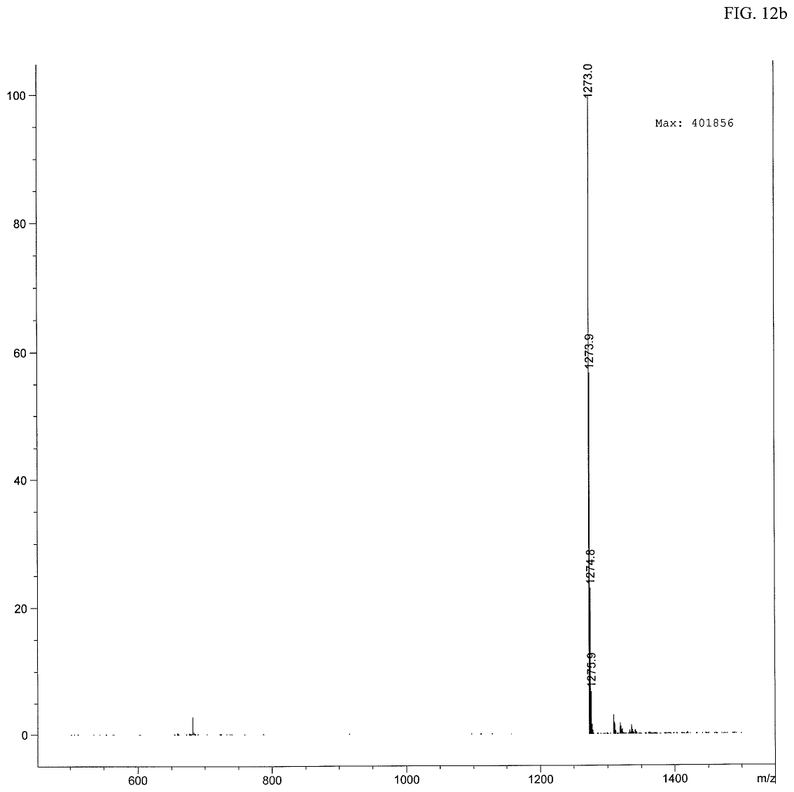

LCMS (FIG. 12a and FIG. 12b) analysis of rebaudioside N2 showed a [M-H]⁻ ion at m/z 1273.0, in good agreement with the expected molecular formula of C₅₆H₉₀O₃₂ (calculated for [C₅₆H₈₉O₃₂]⁻ ion: 1273.5 error <0.05%). The MS data confirms that rebaudioside N2 has a molecular formula of C₅₆H₉₀O₃₂. LCMS analysis was performed in the following conditions listed in Table 6.

TABLE 6
Conditions for LCMS analysis

| Column | Agilent Poroshell 120 SB-C18, | | |
|---|---|---|---|
| | 4.6 mm × 150 mm, 2.7 μm | | |
| Temperature | 40° C. | | |
| Mobile Phase | A: Mobile Phase Premix Solution - | | |
| | 25% Acetonitrile: | | |
| | 75% Formic Acid (0.1% in Water) | | |
| | B: Mobile Phase Premix Solution - | | |
| | 32% Acetonitrile: | | |
| | 68% Formic Acid (0.1% in Water) | | |
| Gradient | Time (min) | A (%) | B (%) |
| | 0 | 100 | 0 |
| | 12.0 | 100 | 0 |
| | 12.5 | 50 | 50 |
| | 13.0 | 0 | 100 |
| | 60.0 | 0 | 100 |

| | |
|---|---|
| Flow rate | 0.5 mL/min |
| Injection | 2 μL |
| Run time | 45 mins |
| Post time | 5 mins |
| Autosampler | Ambient |
| temperature | |
| Detection | MSD at Negative Scan mode |
| MSD Setting | Mode: ES-API, Negative Polarity |
| | Drying gas flow: 13.0 L/min |
| | Nebulizer Pressure: 30 psig |
| | Drying gas temperature: 270° C. |
| | Fragmentor: 50 V |
| | Scan ranges: 500 to 1500 of mass |
| Sample Preparation | 1 mg/ml (30% ACN in water) |

### Example 8

### Structure Elucidation of Rebaudioside O4

NMR experiments were performed on a Bruker 500 MHz spectrometer, with the sample dissolved in pyridine-d5. Along with signals from the sample, signals from pyridine-d5 at δ_{C} 123.5, 135.5, 149.9 ppm and δ_{H} 7.19, 7.55, 8.71 ppm were observed. ¹H-NMR spectrum of rebaudioside O₄ recorded in pyridine-d5 confirmed the excellent quality of the sample (see FIG. 7b). HSQC (see FIG. 8b) shows the presence of an exo-methylene group in the sugar region with a long-range coupling to C-15, observable in the H,H—COSY (FIG. 9b). Other deep-fielded signals of the quaternary carbons (C-13, C-16 and C-19) are detected by the HMBC (FIG. 10b). Correlation of the signals in the HSQC, HMBC and H,H—COSY reveal the presence of steviol glycoside with the following aglycone structure (see FIG. 1e):

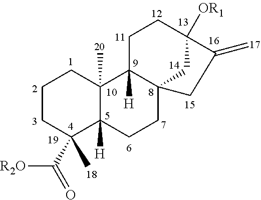

HSQC spectrum of rebaudioside O4 also shows the presence of a methyl-doublet (J=6.3 Hz) with a H,H—COSY correlation to a signal in the sugar region. HSQC also shows the presence of seven anomeric signals, of which six of these anomeric signals have coupling constants of about 8 Hz and the broad signals of their sugar linkage allow the identification of these six sugars as β-D-glucosides. The seventh anomeric signal is a broad singlet and the H,H—COSY correlation to the methyl doublet allow to the identification this sugar as α-L-rhamnose.

The observation of the anomeric protons in combination with HSQC and HMBC reveal the sugar linkage and the correlation to the aglycone. The assignment of the sugar sequence was confirmed by using the combination of HSQC-TOCSY (FIG. 11b) and HSQC.

Results from NMR experiments above were used to assign the chemical shifts of the protons and carbons of the structure of rebaudioside O4 (see Table 7).

TABLE 7
Chemical shifts of rebaudioside O4

| | | | | |
|---|---|---|---|---|
| Position | δ_{C} [ppm] | δ_{H} [ppm] | J [Hz]/(INT) | HMBC (H −> C) |

Aglycone moiety

| 1 | 40.0 | t | 0.67 | m | | |
|---|---|---|---|---|---|---|
| | | | 1.63 | m | | |
| 2 | 19.0 | t | 1.40 | m | | |
| | | | 2.06 | m | | |
| 3 | 37.0 | t | 1.07 | m | | |
| | | | 2.87 | m | | |
| 4 | 44.4 | s | — | | | |
| 5 | 56.9 | d | 0.96 | m | | |
| 6 | 21.3 | t | 1.97 | m | | |
| | | | 2.19 | m | | |
| 7 | 41.3 | t | 1.28 | m | | |
| | | | 1.46 | m | | |
| 8 | 42.1 | s | — | | | |
| 9 | 53.5 | d | 0.84 | m | | |
| 10 | 39.0 | s | — | | | |
| 11 | 20.0 | t | 1.60 | m | | |
| | | | 1.62 | m | | |
| 12 | 38.1 | t | 2.01 | m | | |
| | | | 2.13 | m | | |
| 13 | 86.1 | s | — | | | |
| 14 | 42.6 | t | 1.99 | d | 11.2 | |
| | | | 2.36 | d | 11.2 | |
| 15 | 47.6 | t | 2.01 | d | 15.8 | 7, 8, 9, 14 |
| | | | 2.14 | d | 15.8 | |
| 16 | 153.9 | s | — | | | |
| 17 | 104.5 | t | 5.05 | br s | | 13, 15, 16 |
| | | | 5.62 | br s | | |
| 18 | 28.5 | q | 1.42 | s | (3H) | 3, 4, 5, 19 |
| 19 | 175.3 | s | — | | | |
| 20 | 16.1 | q | 1.01 | s | (3H) | 1, 5, 9, 10 |

Sugar moiety
Sugar I: β-D-Glucopyranoside

| | | | | | | |
|---|---|---|---|---|---|---|
| 1ⁱ | 97.7 | d | 4.92 | d | 7.7 | 13 |
| 2ⁱ | 75.5 | d | 4.22 | m | | |
| 3ⁱ | 91.0 | d | 4.02 | m | | |
| 4ⁱ | 70.1 | d | 4.01 | m | | |
| 5ⁱ | 76.7 | d | 3.55 | m | | |
| 6ⁱ | 70.2 | t | 3.84 | m | | |
| | | | 4.98 | m | | |

Sugar II: α-L-Rhamnopyranoside

| | | | | | | |
|---|---|---|---|---|---|---|
| 1ⁱⁱ | 101.0 | d | 6.50 | br s | | 2ⁱ |
| 2ⁱⁱ | 72.1 | d | 4.81 | m | | |
| 3ⁱⁱ | 72.1 | d | 4.55 | m | | |
| 4ⁱⁱ | 73.2 | d | 4.27 | m | | |
| 5ⁱⁱ | 69.1 | d | 4.87 | m | | |
| 6ⁱⁱ | 18.5 | q | 1.68 | m | 6.3 | |

Sugar III: β-D-Glucopyranoside

| | | | | | | |
|---|---|---|---|---|---|---|
| 1ⁱⁱⁱ | 104.6 | d | 5.02 | d | 8.1 | 3ⁱ |
| 2ⁱⁱⁱ | 74.6 | d | 3.96 | m | | |
| 3ⁱⁱⁱ | 77.9 | d | 4.13 | m | | |
| 4ⁱⁱⁱ | 71.6 | d | 3.91 | m | | |
| 5ⁱⁱⁱ | 78.0 | d | 3.96 | m | | |
| 6ⁱⁱⁱ | 61.8 | t | 4.30 | m | | |
| | | | 4.50 | m | | |

Sugar IV: β-D-Glucopyranoside

| | | | | | | |
|---|---|---|---|---|---|---|
| 1^{iv} | 104.1 | d | 4.76 | d | 7.7 | 6ⁱ |
| 2^{iv} | 75.3 | d | 3.96 | m | | |
| 3^{iv} | 78.1 | d | 4.21 | m | | |
| 4^{iv} | 70.9 | d | 4.27 | m | | |
| 5^{iv} | 77.8 | d | 3.88 | m | | |
| 6^{iv} | 61.8 | t | 4.28 | m | | |
| | | | 4.36 | m | | |

Sugar V: β-D-Glucopyranoside

| | | | | | | |
|---|---|---|---|---|---|---|
| 1^{v} | 92.5 | d | 6.20 | d | 8.1 | 19 |
| 2^{v} | 77.1 | d | 4.47 | m | | |
| 3^{v} | 88.0 | d | 4.22 | m | | |
| 4^{v} | 68.7 | d | 4.14 | m | | |
| 5^{v} | 78.2 | d | 3.79 | m | | |
| 6^{v} | 61.4 | t | 4.20 | m | | |
| | | | 4.33 | m | | |

Sugar VI: βD-Glucopyranoside

| | | | | | | |
|---|---|---|---|---|---|---|
| 1^{vi} | 103.5 | d | 5.75 | d | 7.7 | 2^{vi} |
| 2^{vi} | 75.5 | d | 3.96 | m | | |
| 3^{vi} | 78.1 | d | 4.28 | m | | |
| 4^{vi} | 72.3 | d | 4.08 | m | | |
| 5^{vi} | 78.1 | d | 3.95 | m | | |
| 6^{vi} | 62.8 | t | 4.35 | m | | |
| | | | 4.56 | m | | |

Sugar VII: β-D-Glucopyranoside

| | | | | | | |
|---|---|---|---|---|---|---|
| 1^{vii} | 104.2 | d | 5.31 | d | 7.8 | 3^{v} |
| 2^{vii} | 75.0 | d | 4.06 | m | | |
| 3^{vii} | 77.7 | d | 4.22 | m | | |
| 4^{vii} | 71.3 | d | 4.11 | m | | |
| 5^{vii} | 78.0 | d | 4.01 | m | | |
| 6^{vii} | 62.0 | t | 4.52 | m | | |
| | | | 4.56 | m | | |

Correlation of all NMR results indicates rebaudioside O4 with six β-D-glucoses and one α-L-rhamnose attached to steviol aglycone, as depicted with the following chemical structure (see FIGS. 1c and 1d):

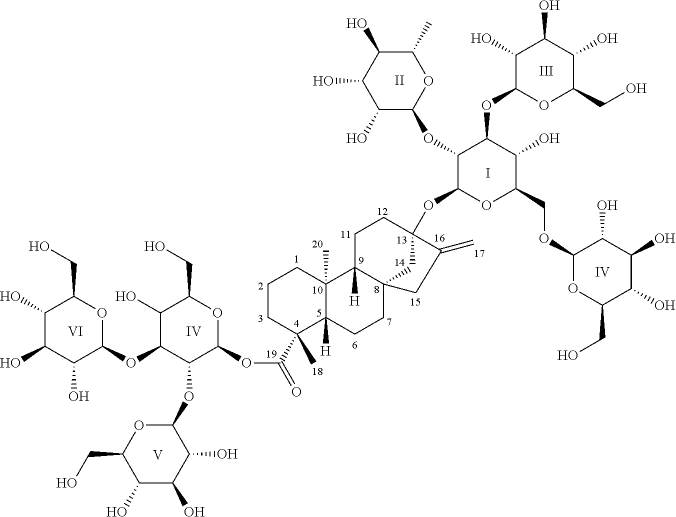

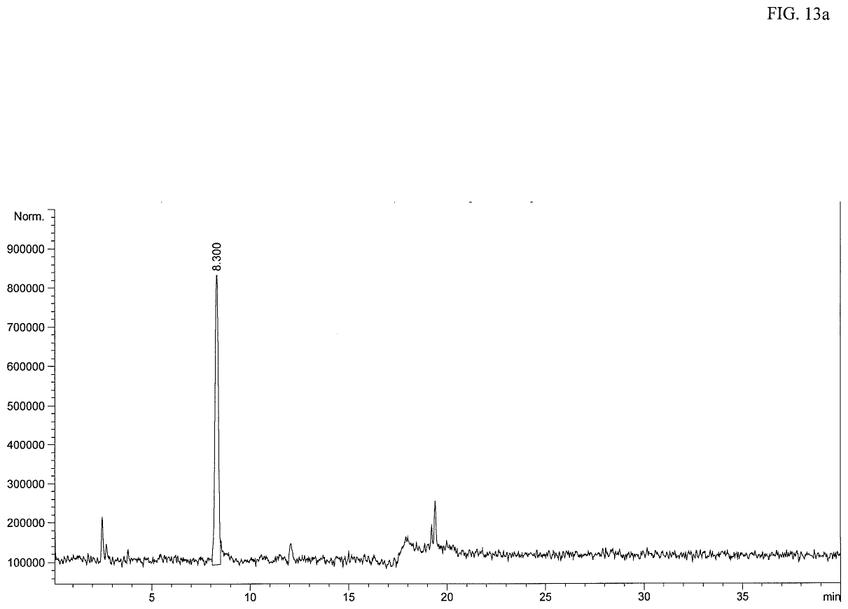

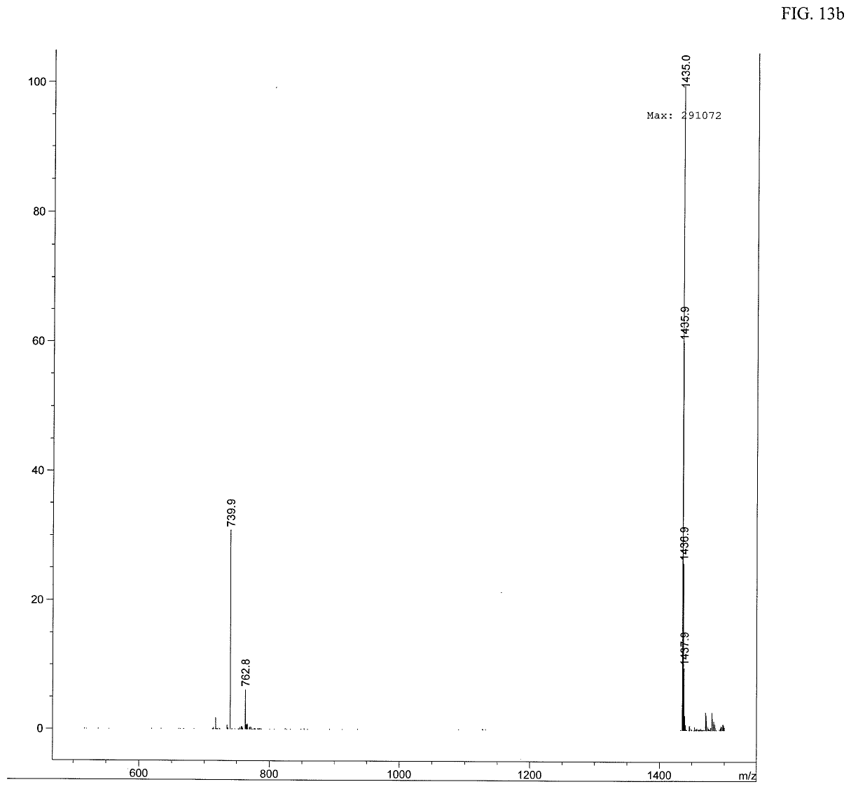

LCMS (FIG. 13a and FIG. 13b) analysis of rebaudioside O4 showed a [M-H]⁻ ion at m/z 1435.0, in good agreement with the expected molecular formula of C₆₂H₁₀₀O₃₇ (calculated for [C₆₂H₉₉O₃₇]⁻ ion: 1435.6, error <0.05%). The MS data confirms that rebaudioside O4 has a molecular formula of C₆₂H₁₀₀O₃₇. LCMS analysis was performed using the conditions listed in Table 6.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. Rebaudioside N2 with the formula:

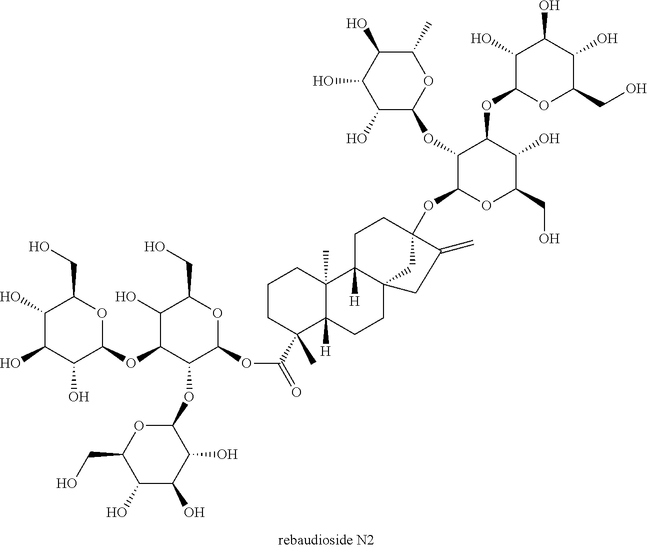

2. Rebaudioside O4 with the formula:

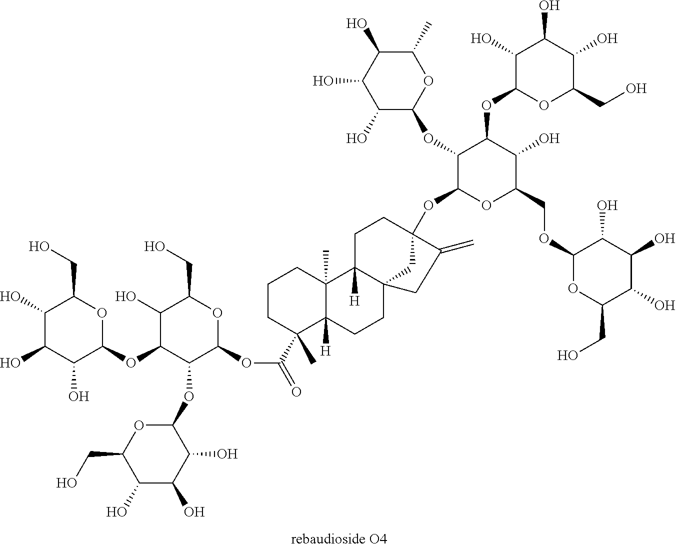

3. A method for producing a highly purified rebaudioside N2 of claim 1, comprising the steps of:
a. providing a starting composition comprising an organic compound with at least one carbon atom;
b. providing an enzyme preparation or microorganism containing at least one enzyme selected from steviol biosynthesis enzymes, UDP-glucosyltransferases, UDP-glycosyltransferases and optionally UDP-glucose and UDP-rhamnose recycling enzymes;
c. contacting the enzyme preparation or microorganism with a medium containing the starting composition to produce a medium comprising rebaudioside N2.

4. A method for producing a highly purified rebaudioside O4 of claim 2, comprising the steps of:
a. providing a starting composition comprising an organic compound with at least one carbon atom;
b. providing an enzyme preparation or microorganism containing at least one enzyme selected from steviol biosynthesis enzymes, UDP-glucosyltransferases, UDP-glycosyltransferases and optionally UDP-glucose and UDP-rhamnose recycling enzymes;
c. contacting the enzyme preparation or microorganism with a medium containing the starting composition to produce a medium comprising rebaudioside O4.

5. A method for producing a highly purified rebaudioside N2 of claim 1, comprising the steps of:
a. providing a starting composition comprising an organic compound with at least one carbon atom;
b. providing a biocatalyst comprising at least one enzyme selected from steviol biosynthesis enzymes, UDP-glucosyltransferases, UDP-glycosyltransferases and optionally UDP-glucose and UDP-rhamnose recycling enzymes;
c. contacting the biocatalyst with a medium containing the starting composition to produce a medium comprising rebaudioside N2.

6. A method for producing a highly purified rebaudioside O4 of claim 2, comprising the steps of:
a. providing a starting composition comprising an organic compound with at least one carbon atom;
b. providing a biocatalyst comprising at least one enzyme selected from steviol biosynthesis enzymes, UDP-glucosyltransferases, UDP-glycosyltransferases and optionally UDP-glucose and UDP-rhamnose recycling enzymes;
c. contacting the biocatalyst with a medium containing the starting composition to produce a medium comprising rebaudioside O4.

7. The method of claim 3 further comprising the step of:
d. separating the rebaudioside N2 from the medium to provide a highly purified rebaudioside N2 composition.

8. (canceled)

9. The method of claim 3, wherein the starting composition is selected from the group consisting of steviol, steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3 other steviol glycosides, polyols, carbohydrates, and combinations thereof.

10. The method of claim 3, wherein the microorganism is selected from the group consisting of E. coli, Saccharomyces sp., Aspergillus sp., Pichia sp., Bacillus sp., and Yarrowia sp.

11. The method of claim 5, wherein the biocatalyst is an enzyme, or a cell comprising one or more enzyme, capable of converting the starting composition to rebaudioside N2.

12. (canceled)

13. The method of claim 3, or wherein the enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGT91D2, EUGT11, UGTSl2, UGT76G1, UGlyT91C1 or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity.

14. The method of claim 7, wherein the rebaudioside N2 content in highly purified rebaudioside N2 composition is greater than about 95% by weight on a dry basis.

15. (canceled)

16. A consumable product comprising rebaudioside N2 of claim 1, wherein the product is selected from the group consisting of a food, a beverage, a pharmaceutical composition, a tobacco product, a nutraceutical composition, an oral hygiene composition, and a cosmetic composition.

17. (canceled)

18. The consumable product of claim 16 or 17, wherein the product is selected from the group consisting of beverages; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco and medicinal products.

19. The consumable product of claim 16 or 17, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers and combinations thereof.

20. The consumable product of claim 16, further comprising at least one functional ingredient selected from the group consisting of saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

21. The consumable product of claim 16 or 17, further comprising a compound selected from the group consisting of steviolmonoside, steviolmonoside A, steviolbioside A, steviolbioside B, steviolbioside C, steviolbioside D, steviolbioside E, rubusoside, dulcoside A, dulcoside C, dulcoside D, stevioside A, stevioside B, stevioside C, stevioside G, stevioside H, rebaudioside B2, rebaudioside A4, rebaudioside C, rebaudioside C3, rebaudioside C4, rebaudioside C5, rebaudioside C6, rebaudioside E3, rebaudioside E4, rebaudioside E5, rebaudioside E6, rebaudioside E7, rebaudioside D5, rebaudioside D6, rebaudioside D7, rebaudioside D8, rebaudioside H2, rebaudioside H3, rebaudioside H4, rebaudioside H5, rebaudioside H6, rebaudioside K, rebaudioside N2, rebaudioside N3, rebaudioside N4, rebaudioside N5, rebaudioside M3, rebaudioside A, rebaudioside A2, rebaudioside A3, rebaudioside AM, rebaudioside B, rebaudioside C2, rebaudioside D, rebaudioside D2, rebaudioside D3, rebaudioside D4, rebaudioside E, rebaudioside E2, rebaudioside F, rebaudioside F2, rebaudioside F3, rebaudioside G, rebaudioside H, rebaudioside I, rebaudioside 12, rebaudioside 13, rebaudioside J, rebaudioside K2, rebaudioside L, rebaudioside M, rebaudioside M2, rebaudioside N, rebaudioside O, rebaudioside O2, rebaudioside O3, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside W, rebaudioside W2, rebaudioside W3, rebaudioside Y, rebaudioside Z1, rebaudioside Z2, steviolbioside, stevioside, stevioside D, stevioside E, stevioside E2, stevioside F, NSF-02, Mogroside V, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose.

22. A method for enhancing the sweetness of a beverage or food product, comprising a sweetener comprising:
a. providing a beverage or food product comprising a sweetener; and
b. adding a sweetness enhancer comprising rebaudioside N2 of claim 1,
wherein rebaudioside N2 is present in a concentration at or below the sweetness recognition threshold.

23. (canceled)

24. A method for modifying the flavor of a beverage or food product, comprising
a. providing a beverage or food product, and
b. adding a composition comprising rebaudioside N2 of claim 1.

25. (canceled)

26. A method for suppressing foaming of a beverage or food product, comprising
a. providing a beverage or a food product, and
b. adding a foam suppressor comprising rebaudioside N2 of claim 1.

27. (canceled)


SEQUENCE LISTING (4 sequences)

SEQ ID NO: 1 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 442 aa, solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 3 - protein, 458 aa, Stevia rebaudiana
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL

SEQ ID NO: 4 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD